(19) United States
(12) Patent Application Publication
(10) Pub. No.: US 2017/0332963 A1
(43) Pub. Date: Nov. 23, 2017

(54) BLOOD PRESSURE MEASUREMENT DEVICE

(71) Applicant: Panasonic Intellectual Property Management Co., Ltd., Osaka (JP)

(72) Inventors: KENTA MURAKAMI, Osaka (JP); MOTOTAKA YOSHIOKA, Osaka (JP); JUN OZAWA, Nara (JP)

(21) Appl. No.: 15/589,006

(22) Filed: May 8, 2017

(30) Foreign Application Priority Data
May 19, 2016 (JP) 2016-100818

(51) Int. Cl. A61B 5/00; A61B 5/1171; A61B 5/021

(52) U.S. Cl. CPC A61B 5/681; A61B 5/7278; A61B 5/02125; A61B 5/7445; A61B 5/0077; A61B 5/684; A61B 5/1176; A61B 2562/0233; A61B 2576/00

(57) ABSTRACT

A blood pressure measurement device includes an annular bracelet that comes into contact with the user's wrist; a display that is disposed on the outer surface of the bracelet and includes a display surface; a camera that is disposed on the outer surface of the bracelet, has an optical axis tilted from the direction of a normal to the display surface, and captures images of the user; a pulse wave sensor that is disposed on an inner surface of the bracelet and detects a pulse wave at the user's wrist; and a processing circuit that estimates the user's blood pressure. The processing circuit calculates a first pulse wave timing from a temporal change in luminance in the cheek region in the images, determines a second pulse wave timing from the detected pulse wave, and estimates the blood pressure from a time difference between the first and second pulse wave timings.

### BACKGROUND

### 1. Technical Field

The present disclosure relates to a blood pressure measurement device that is wearable on the wrist of a user and that measures blood pressure by using images of the user.

### 2. Description of the Related Art

International Publication No. 2014/136310 discloses a device that measures blood pressure or pulse rate by using images of the user's face and hand that have been captured with a camera of a smartphone or the like.

In addition, Japanese Unexamined Patent Application Publication No. 2012-12581 discloses a wristwatch-type device that calculates blood pressure by using a pulse wave of a user that is acquired by the device and an electrocardiogram acquired as a result of the user touching the device.

The technique of the related art disclosed in International Publication No. 2014/136310 requires the user to capture images each including both the hand and the face with a camera in order to measure blood pressure. Consequently, the user needs to position their hand by their face to capture images, that is, the user is required to take an unnatural pose.

In addition, the technique of the related art disclosed in Japanese Unexamined Patent Application Publication No. 2012-12581 requires the user to touch the device (sensor) with the hand on the side opposite to the side where the device is worn, in order to measure blood pressure. Thus, such a requirement becomes burdensome to the user every time blood pressure measurement is performed.

### SUMMARY

One non-limiting and exemplary embodiment provides a wristwatch-type blood pressure measurement device capable of measuring blood pressure relatively easily.

In one general aspect, the techniques disclosed here feature a blood pressure measurement device including a bracelet that comes into contact with a wrist of a user, the bracelet having an annular shape and having an outer surface and an inner surface; a display that is disposed on the outer surface of the bracelet and that includes a display surface; an image capturing device that is disposed on the outer surface of the bracelet and that captures a plurality of images of the user, the image capturing device having an optical axis that is tilted with respect to a direction of a normal to the display surface of the display; a pulse wave detector that is disposed on the inner surface of the bracelet and that detects a pulse wave at the wrist of the user; and a processing circuit that estimates a blood pressure of the user, wherein the processing circuit calculates a first pulse wave timing from a temporal change in luminance value in a cheek region of the user in the plurality of images, determines a second pulse wave timing from the pulse wave detected by the pulse wave detector, and estimates a blood pressure of the user from a time difference between the first pulse wave timing and the second pulse wave timing.

According to the general aspect of the present disclosure, blood pressure can be measured relatively easily.

It should be noted that general or specific embodiments may be implemented as a system, a method, an integrated circuit, a computer program, a computer-readable recording medium, or any selective combination thereof. Examples of the computer-readable recording medium include a nonvolatile recording medium, for example, Compact Disc-Read Only Memory (CD-ROM).

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a schematic view of a blood pressure measurement device according to the underlying knowledge forming the basis of the present disclosure;

FIG. 1B is a schematic view of a blood pressure measurement device according to the underlying knowledge forming the basis of the present disclosure;

FIG. 2 is a diagram illustrating a usage example of the blood pressure measurement device according to the underlying knowledge forming the basis of the present disclosure;

FIG. 3 is a diagram illustrating a usage example of a blood pressure measurement device according to a first embodiment;

FIG. 4A is a plan view of the blood pressure measurement device according to the first embodiment;

FIG. 4B is a bottom plan view of the blood pressure measurement device according to the first embodiment;

FIG. 4C is a cross-sectional view of the blood pressure measurement device according to the first embodiment;

FIG. 4D is a diagram illustrating a situation in which a pulse wave sensor detects a pulse wave in accordance with the first embodiment;

FIG. 5A is a diagram illustrating a positional relationship between the blood pressure measurement device according to the first embodiment and the user's face;

FIG. 5B is a diagram illustrating a positional relationship between the blood pressure measurement device according to the first embodiment and the user's face;

FIG. 6 is a block diagram illustrating a functional configuration of the blood pressure measurement device according to the first embodiment;

FIG. 7 is a flowchart illustrating a process performed by the blood pressure measurement device according to the first embodiment;

FIG. 8A is a diagram illustrating a user's biomechanics model used in the blood pressure measurement device according to the first embodiment;

FIG. 8B is a diagram illustrating a user's biomechanics model used in the blood pressure measurement device according to the first embodiment;

FIG. 9A is a diagram for describing how an instruction is given to the user by the blood pressure measurement device according to the first embodiment;

FIG. 9B is a diagram illustrating a display example of an instruction to the user in accordance with the first embodiment;

FIG. 9C is a diagram illustrating a display example of an instruction to the user in accordance with the first embodiment;

FIG. 10A is a diagram for describing a method for determining a region in an image in accordance with the first embodiment;

FIG. 10B is a diagram for describing a method for determining a region in an image in accordance with the first embodiment;

FIG. 11 is a diagram illustrating a method for determining a region in an image on the basis of one of the eyes, one of the ears, and the nose in accordance with the first embodiment;

FIG. 12 is a diagram illustrating temporal changes in luminance values for red (R), green (G), and blue (B) in a cheek region in images of the face;

FIG. 13A is a diagram for describing how a peak in the waveform of a pulse wave is detected in accordance with the first embodiment;

FIG. 13B is a diagram for describing how peaks in the waveform of a pulse wave are detected in accordance with the first embodiment;

FIG. 14 is a diagram for describing the fact that pulse wave timings calculated from images correlate to the actual pulse wave;

FIG. 15 is a diagram for describing how blood pressure is estimated in accordance with the first embodiment;

FIG. 16A is a diagram illustrating an example of information displayed on a display unit in accordance with the first embodiment;

FIG. 16B is a diagram illustrating an example of information displayed on the display unit in accordance with the first embodiment;

FIG. 17A is a diagram illustrating another example of information displayed on the display unit in accordance with the first embodiment;

FIG. 17B is a diagram illustrating another example of information displayed on the display unit in accordance with the first embodiment;

FIG. 17C is a diagram illustrating another example of information displayed on the display unit in accordance with the first embodiment;

FIG. 18 is a diagram illustrating an example of how an instruction is given to the user by a blood pressure measurement device according to a second embodiment;

FIG. 19 is a diagram illustrating an example of how an instruction is given to the user by the blood pressure measurement device according to the second embodiment;

FIG. 20 is a diagram illustrating an example of how an instruction is given to the user by a blood pressure measurement device according to a third embodiment;

FIG. 21 is a diagram illustrating an example of how an instruction is given to the user by the blood pressure measurement device according to the third embodiment;

FIG. 22 is a diagram illustrating an example of how information is provided to the user by a blood pressure measurement device according to another embodiment;

FIG. 23A is a graph illustrating a temporal change in luminance of cheek images;

FIG. 23B is a graph of the first derivative of the temporal change in luminance of the images;

FIG. 24A is a diagram illustrating a display example of blood pressure and pulse rate in accordance with another embodiment;

FIG. 24B is a diagram illustrating a display example of blood pressure and pulse rate in accordance with another embodiment;

FIG. 24C is a diagram illustrating a display example of blood pressure and pulse rate in accordance with another embodiment;

FIG. 25A is a diagram illustrating a display example of blood pressure and pulse rate in accordance with another embodiment;

FIG. 25B is a diagram illustrating a display example of blood pressure and pulse rate in accordance with another embodiment;

FIG. 25C is a diagram illustrating a display example of blood pressure and pulse rate in accordance with another embodiment;

FIG. 26 is a diagram illustrating feature points of waveforms of pulse waves extracted from a plurality of regions in accordance with another embodiment;

FIG. 27 is a diagram illustrating a relationship between the user's body position and the propagation route of the pulse wave in accordance with another embodiment;

FIG. 28 is a diagram for describing how differential pulse transit time is corrected on the basis of the user's body position in accordance with another embodiment;

FIG. 29A is a diagram illustrating the disposed position of the camera in accordance with another embodiment;

FIG. 29B is a diagram illustrating the disposed position of the camera in accordance with another embodiment; and

FIG. 30 is a diagram illustrating a curved screen display in another embodiment.

### DETAILED DESCRIPTION

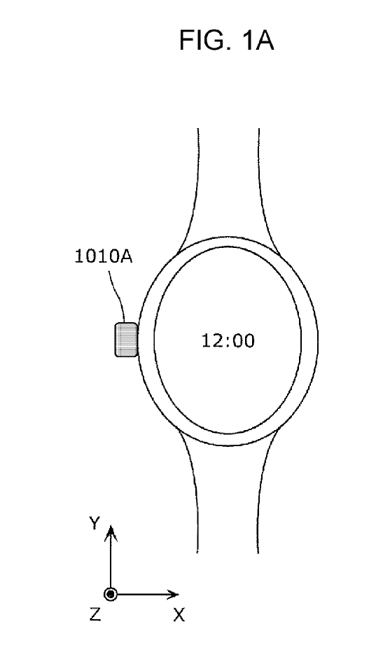

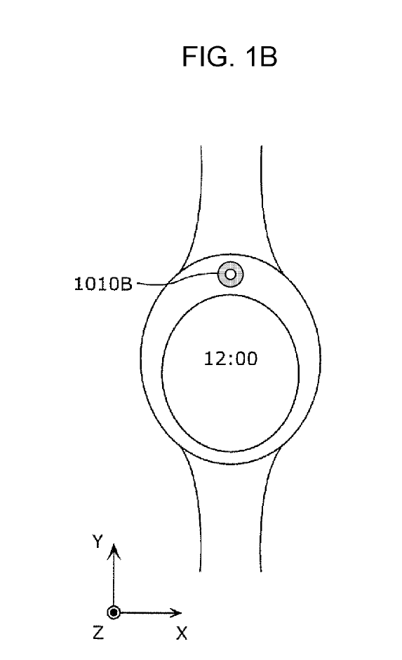

Underlying Knowledge Forming Basis of the Present Disclosure Cameras of wristwatch-type devices are typically intended to capture images of sceneries, people other than the user, or objects. Accordingly, a camera 1010A and a camera 1010B are disposed on the side of the display and above the display as illustrated in FIGS. 1A and 1B, respectively.

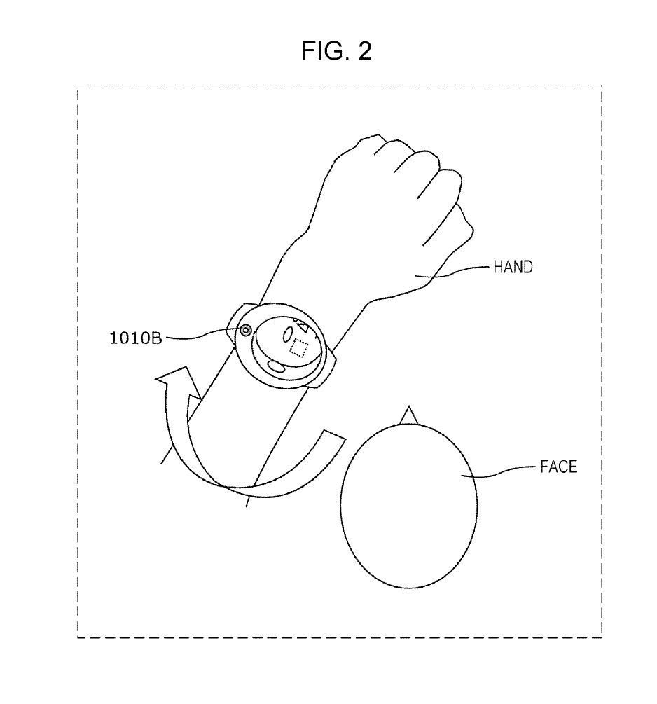

For example, in the case where the camera 1010B is disposed above the display of a blood pressure measurement device as illustrated in FIG. 1B, it is difficult to capture an image of the user's face unless the user move their body by a large amount to look into the blood pressure measurement device as illustrated in FIG. 2 when the user measures blood pressure.

To cope with such an inconvenience, a blood pressure measurement device according to an aspect of the present disclosure includes a bracelet that comes into contact with a wrist of a user, the bracelet having an annular shape and having an outer surface and an inner surface; a display that is disposed on the outer surface of the bracelet and that includes a display surface; an image capturing device that is disposed on the outer surface of the bracelet and that captures a plurality of images of the user, the image capturing device having an optical axis that is tilted with respect to a direction of a normal to the display surface of the display; a pulse wave detector that is disposed on the inner surface of the bracelet and that detects a pulse wave at the wrist of the user; and a processing circuit that estimates a blood pressure of the user, wherein the processing circuit calculates a first pulse wave timing from a temporal change in luminance value in a cheek region of the user in the plurality of images, determines a second pulse wave timing from the pulse wave detected by the pulse wave detector, and estimates a blood pressure of the user from a time difference between the first pulse wave timing and the second pulse wave timing.

With this configuration, the optical axis of the image capturing device is successfully tilted with respect to the direction of the normal to the display surface. This arrangement consequently makes it easier to capture an image of a portion of the user's body suitable for calculation of the first pulse wave timing and can increase the blood pressure estimation accuracy.

In addition, in the blood pressure measurement device according to the aspect of the present disclosure, the display surface of the display may have a top-bottom direction and a left-right direction, and when viewed from the top-bottom direction of the display surface of the display, the optical axis of the image capturing device may be tilted in the left-right direction of the display surface of the display with respect to the direction of the normal.

With this configuration, the optical axis of the image capturing device is successfully tilted in the left-right direction of the display surface with respect to the direction of the normal to the display surface. This arrangement consequently makes it easier to capture an image of a region for use in calculation of the first pulse wave timing and can increase the blood pressure estimation accuracy.

In addition, in the blood pressure measurement device according to the aspect of the present disclosure, the processing circuit may further determine the cheek region of the user in the plurality of images of the user, and calculate the first pulse wave timing in accordance with a temporal change in luminance value in the determined cheek region.

With this configuration, the first pulse wave timing is successfully calculated on the basis of a temporal change in luminance value in a cheek region, which can consequently increase the blood pressure estimation accuracy.

In addition, in the blood pressure measurement device according to the aspect of the present disclosure, the processing circuit may further determine whether the cheek region of the user is successfully determined in the plurality of images of the user, calculate a relative position of a face of the user with respect to the image capturing device by using the plurality of images of the user upon failing to determine the cheek region of the user, and display on the display an instruction for changing a positional relationship between the face of the user and the image capturing device in accordance with the relative position of the face of the user.

With this configuration, an instruction for changing the positional relationship between the user's face and the image capturing device is successfully displayed on a display on the basis of the relative position of the user's face when the user's cheek region is not determined in images. Such an instruction allows the user to appropriately change the positional relationship between their face and the image capturing device, and consequently an image of the user's cheek region can be captured easily.

In addition, in the blood pressure measurement device according to the aspect of the present disclosure, the processing circuit may calculate the relative position of the face of the user on the basis of a size and a position of at least one of an eye, an ear, and a nose of the user in the plurality of images of the user.

With this configuration, the relative position of the user's face can be calculated relatively easily on the basis of the size and position of at least one of an eye, an ear, and a nose of the user.

In addition, in the blood pressure measurement device according to the aspect of the present disclosure, the processing circuit may further select a recognition model, from among a plurality of recognition models used to recognize at least one of the eye, the ear, and the nose of the user in images, on the basis of which of a right wrist and a left wrist of the user the blood pressure measurement device is worn on and which of a palm side and a back-of-hand side of the wrist the blood pressure measurement device is worn on, and recognize at least one of the eye, the ear, and the nose of the user in the plurality of images of the user by using the selected recognition model.

With this configuration, recognition models can be switched between in accordance with the position where the blood pressure measurement device is worn. Thus, the relative position of the user's face can be calculated more accurately, and consequently a more appropriate instruction can be displayed.

In addition, in the blood pressure measurement device according to the aspect of the present disclosure, the processing circuit may determine which of the palm side and the back-of-hand side the blood pressure measurement device is worn on, on the basis of a temporal change in position of at least one of the eye, the ear, and the nose of the user in the plurality of images of the user.

With this configuration, it is successfully determined which of the palm side and the back-of-hand side the blood pressure measurement device is worn on, on the basis of a temporal change in the position of at least one of the eye, the ear, and the nose in images. Thus, the blood pressure measurement device can automatically determine the position where the blood pressure measurement device is worn, and consequently inputting of the position of the blood pressure measurement device or the like by the user can be omitted.

In addition, in the blood pressure measurement device according to the aspect of the present disclosure, the processing circuit may calculate a distance and an orientation of the face of the user with respect to the image capturing device on the basis of the sizes and positions of the eye, the ear, and the nose of the user in the plurality of images of the user, and display on the display at least one of an instruction for twisting the wrist and an instruction for bending or stretching an elbow in accordance with the calculated distance and orientation.

With this configuration, at least one of an instruction for twisting the wrist and an instruction for bending or stretching the elbow can be displayed on the display. Thus, the user can make a move in accordance with an intuitive and easy-to-understand instruction, and it becomes easier to adjust the relative position of the image capturing device.

In addition, in the blood pressure measurement device according to the aspect of the present disclosure, the processing circuit may control, in accordance with a relative position of the face of the user, at least one of a display angle, a display position, and a display size of information displayed on the display.

With this configuration, at least one of the display angle, the display position, and the display size of information can be controlled in accordance with the relative position of the user's face. Thus, the relative position of the user's face is successfully controlled through a move of the user to see the information, and consequently an image of the user that is more suitable for estimation of blood pressure can be captured.

In addition, in the blood pressure measurement device according to the aspect of the present disclosure, the processing circuit may reduce the display size of the information displayed on the display when the distance of the face of the user with respect to the image capturing device is greater than a threshold distance.

With this configuration, the display size of the information can be reduced when the distance of the user's face from the image capturing device is greater than a predetermined distance threshold. This arrangement consequently causes the user to bring their face closer to the display and the image capturing device in order to see the information.

In addition, in the blood pressure measurement device according to the aspect of the present disclosure, the processing circuit may further determine whether the user is backlit on the basis of luminance values of the plurality of images of the user, and display on the display an instruction for moving at least one of the image capturing device and the face of the user upon determining that the user is backlit.

With this configuration, an instruction for moving at least one of the image capturing device and the user's face can be displayed on the display when the user is backlit. Consequently, the issue regarding backlighting is successfully resolved, and an image more suitable for estimation of blood pressure can be captured.

In addition, in the blood pressure measurement device according to the aspect of the present disclosure, the processing circuit may display on the display an instruction for moving the blood pressure measurement device to an upper position upon determining that the user is backlit.

With this configuration, an instruction for moving the blood pressure measurement device to an upper position can be displayed on the display when the user is backlit. Accordingly, the issue regarding backlighting is successfully resolved when the light source is located above the user, and an image more suitable for estimation of blood pressure can be captured.

In addition, in the blood pressure measurement device according to the aspect of the present disclosure, the processing circuit may display on the display an instruction for twisting a body of the user upon determining that the user is backlit.

With this configuration, an instruction for twisting the user's body is displayed on the display when the user is backlit. Accordingly, the issue regarding backlighting is successfully resolved when the light source is located on the side of the user, and an image more suitable for estimation of blood pressure can be captured.

It should be noted that general or specific embodiments may be implemented as a system, a method, an integrated circuit, a computer program, a computer-readable recording medium such as a CD-ROM, or any selective combination thereof.

Embodiments will be described below with reference to the accompanying drawings.

Note that each embodiment to be described below provides general or specific examples. The values, shapes, materials, components, arrangement and connection of the components, steps, the order of steps, etc., described in the following embodiments are merely illustrative and are not intended to limit the claims. Among the components in the following embodiments, a component not recited in any of the independent claims indicating the most generic concept is described as an optional component.

In addition, each drawing is a schematic drawing and is not necessarily a precise illustration. Further, the same or substantially the same components are denoted by the same reference sign in the drawings. In the following embodiments, the expression accompanying “substantially”, such as “substantially the same”, is sometimes used. For example, the expression “substantially the same” not only indicates the state of being completely the same but also indicates the state of being substantially the same, that is, the state where an error of several percent, for example, is allowed.

### First Embodiment

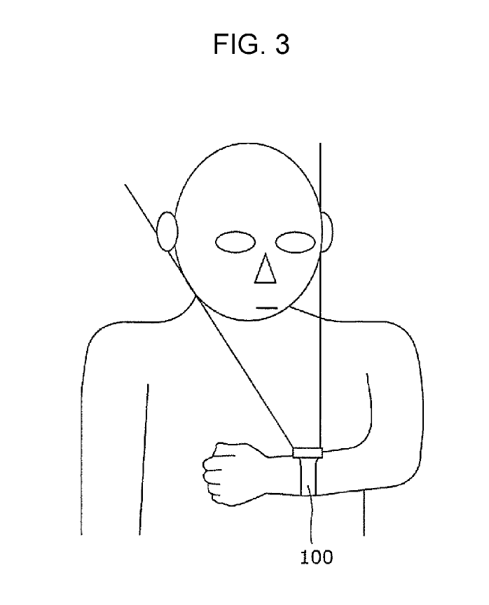

A wristwatch-type blood pressure measurement device 100 according to a first embodiment will be described. FIG. 3 illustrates a usage example of the wristwatch-type blood pressure measurement device 100 according to the first embodiment. As illustrated in FIG. 3, the blood pressure measurement device 100 is worn on the user's wrist, captures images of at least part of the user's face in that state, and measures the user's blood pressure by using the images of the at least part of the user's face.

### Structure of Blood Pressure Measurement Device

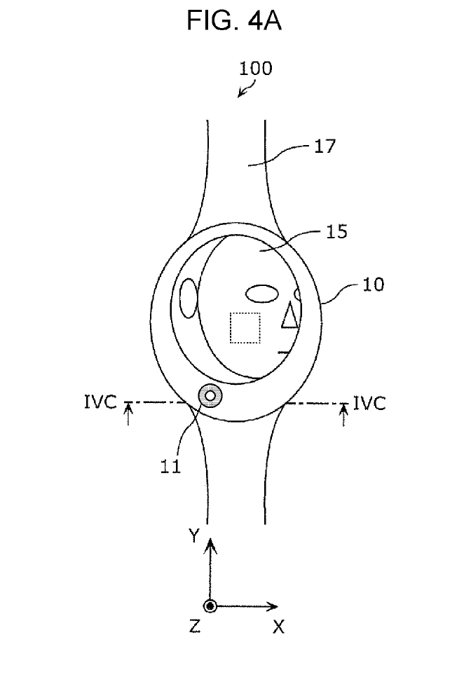

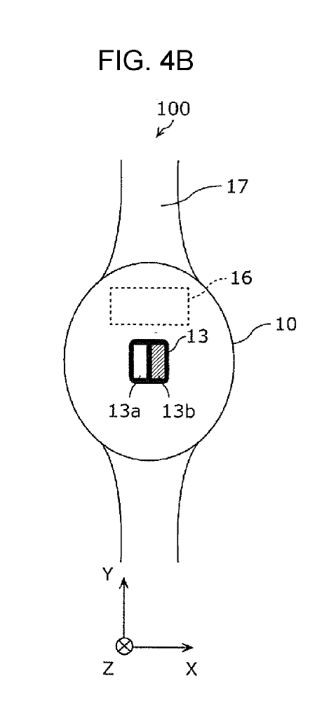

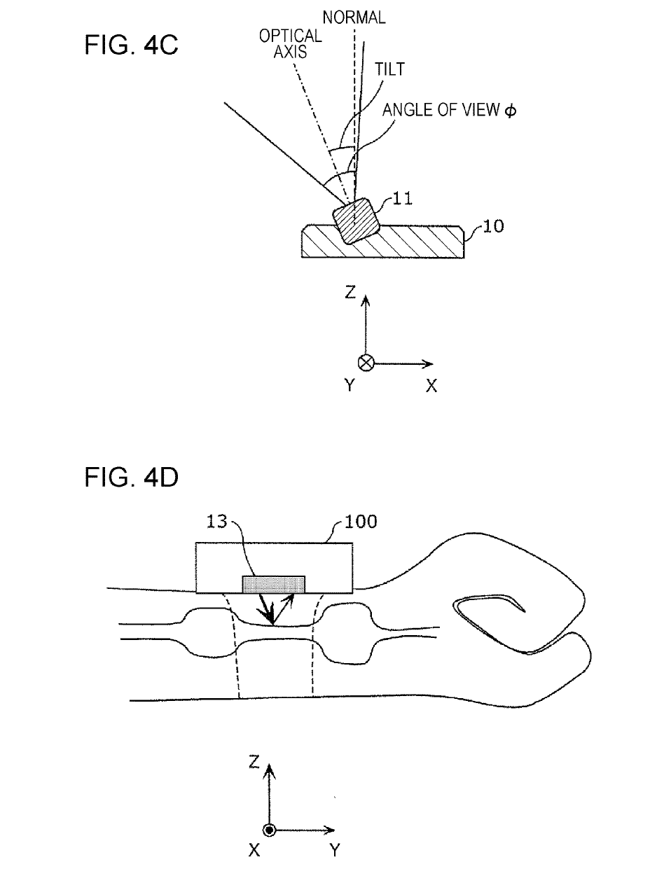

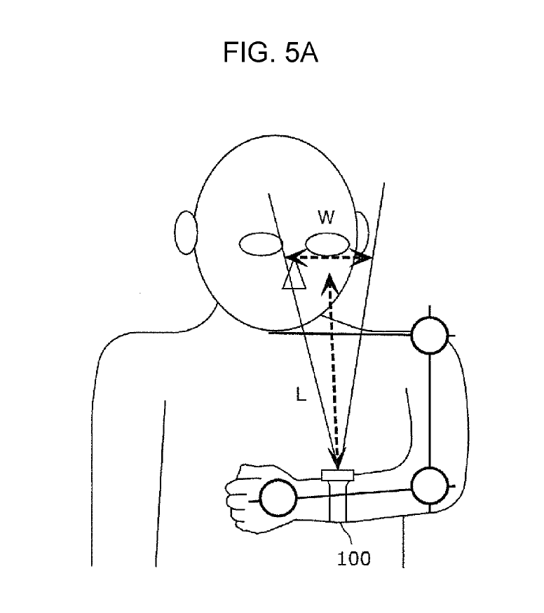

The structure of the blood pressure measurement device 100 according to the first embodiment will be described with reference to FIGS. 4A to 4C. FIG. 4A is a plan view of the blood pressure measurement device 100 according to the first embodiment. That is, FIG. 4A illustrates the outer surface of the blood pressure measurement device 100. FIG. 4B is a bottom plan view of the blood pressure measurement device 100 according to the first embodiment. That is, FIG. 4B illustrates the inner surface of the blood pressure measurement device 100 according to the first embodiment. FIG. 4C is a cross-sectional view of the blood pressure measurement device 100 according to the first embodiment. Specifically, FIG. 4C is a cross-sectional view that is taken along line IVC-IVC illustrated in FIG. 4A and is viewed from the bottom of a display surface of a display 15 in FIG. 4A.

In the following drawings, the left-right direction and the top-bottom direction of the display surface of the display 15 are respectively denoted by the X direction and the Y direction, and the direction of the normal to the display surface of the display 15 is denoted by the Z direction.

The blood pressure measurement device 100 includes a casing 10 and a band 17. The casing 10 and the band 17 constitute an annular bracelet that comes into contact with the wrist of the user.

### Casing

The casing 10 is made of a metal or a resin. The band 17 is attached to the casing 10. The casing 10 includes a camera 11, a pulse wave sensor 13, the display 15, and a processing circuit 16.

### Camera

The camera 11 is disposed on the outer surface of the casing 10. The outer surface of the casing 10 is a surface that is opposite to an inner surface that comes into contact with the wrist of the user when the blood pressure measurement device 100 is worn on the wrist of the user. The outer surface of the casing 10 is a portion of the outer surface of the bracelet.

In the first embodiment, the camera 11 is disposed on the lower left side of the display surface of the display 15 on the outer surface of the casing 10 as illustrated in FIG. 4A. Specifically, the camera 11 is disposed at the position of 7 o'clock with respect to the center of the display 15.

When viewed from the top-bottom direction (Y direction) of the display surface of the display 15, the optical axis of the camera 11 is tilted in the left-right direction (X direction) of the display surface of the display 15 with respect to the normal (Z axis) to the display surface of the display 15 as illustrated in FIG. 4C. For example, when the user wears the blood pressure measurement device 100 on the back-of-hand side of the left wrist, the optical axis of the camera 11 is tilted toward the left side of the user with respect to the direction of the normal to the display surface of the display 15. That is, the optical axis of the camera 11 is tilted toward the left side of the display 15 with respect to the normal to the display surface of the display 15 in order to capture images of a cheek region of the user's face. The left side of the display 15 is the left side of text displayed on the display 15. More specifically, the optical axis of the camera 11 is tilted with respect to the direction of the normal to the display surface of the display 15 at an angle ranging from 4 degrees to 23 degrees, for example.

The camera 11 captures images of a cheek region of the user's face. A pulse wave is detected from the captured images. The camera 11 may also determine whether the user's cheek is included in an image capturing range and may notify the user of the determined result. The camera 11 includes, for example, a charge coupled device (CCD) image sensor or a complementary metal oxide semiconductor (CMOS) image sensor.

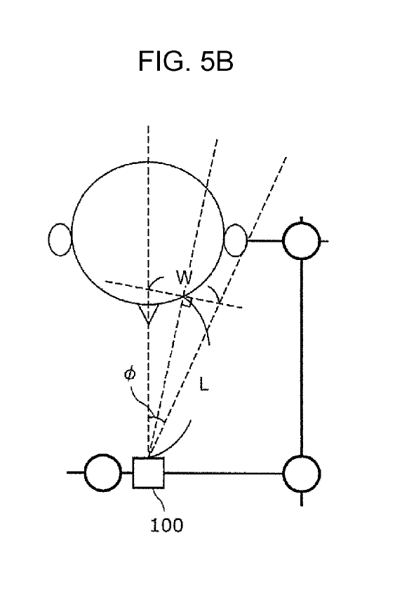

In general, cameras used to capture an image of a user in an application, such as a videotelephony application, may be wide-angle cameras so that the user is entirely included in the image capturing range. In addition, for example, in the case of cameras of mobile phones, the image capturing unit is sometimes tilted, for example, downward to make it easier for the users to capture an image of themselves. However, since the camera 11 according to the first embodiment is intended to detect a pulse wave from a change in luminance in a skin region, it is desirable that an image of a skin region be captured more accurately in a zoom-in state, and the entire face or background need not necessarily be included in the image. For example, regarding the positional relationship between the cheek and the arm when the user looks at the display 15, the degree of zoom or the angle of view of the camera 11 is set such that at least the nose and one of the ears, instead of the entire face, are included in the image capturing range as illustrated in FIGS. 5A and 5B. In general, a distance L is in a range from approximately 10 cm to approximately 40 cm, whereas a width W is in a range from approximately 10 cm to approximately 20 cm in FIGS. 5A and 5B. At that time, the optical axis of the camera 11 is tilted by approximately 4 degrees to 23 degrees with respect to the direction of the normal to the display surface of the display 15. In this way, an image of the user's cheek is effectively captured. As described above, the blood pressure measurement device 100 has a feature in which the image capturing unit is tilted so that the user can take an image of their cheek region by just looking at the display unit of the blood pressure measurement device 100, instead of taking an image of the entire face. With such a configuration, an image of the cheek region suitable for extraction of a pulse wave is successfully captured, and consequently the blood pressure estimation accuracy increases.

The angle of the optical axis of the camera 11 is determined in accordance with the distance L between the cheek and the arm and the width W between the nose and the ear. Since the angle of view φ corresponds to the vertex angle of an isosceles triangle having the height of L and the base of W as illustrated in FIG. 5B, the angle of view φ is denoted by (Equation 1).

$$φ = 2 {tan}^{- 1} \frac{W}{2 L} \qquad ( Equation 1 )$$

The tilt angle of the optical axis of the camera 11 is equal to half the angle of view φ. Accordingly, in the case where the distance L is in a range from 10 cm to 40 cm and the width W is in a range from 10 cm to 20 cm, the tilt angle of the optical axis of the camera 11 is greater than or equal to 4 degrees and less than or equal to 23 degrees.

If the tilt angle of the optical axis of the camera 11 is less than 4 degrees or greater than 23 degrees, the cheek region no longer fits within the imaging capturing range of the camera 11 and it becomes difficult to detect a pulse wave used to estimate blood pressure.

### Pulse Wave Sensor

The pulse wave sensor 13 is disposed on the inner surface of the casing 10. The inner surface of the casing 10 is a surface that comes into contact with the user's wrist when the blood pressure measurement device 100 is worn on the user's wrist. The inner surface of the casing 10 is a portion of the inner surface of the bracelet.

The pulse wave sensor 13 detects a pulse wave of the user at the user's wrist. In the first embodiment, the pulse wave sensor 13 is a photoplethysmography sensor. The pulse wave sensor 13 includes a light-emitter unit 13a and a light-detector unit 13b as illustrated in FIG. 4B.

The light-emitter unit 13a is, for example, a light-emitting diode (LED). The light-emitter unit 13a emits green light.

The light-detector unit 13b is, for example, a photodetector. The light-detector unit 13b detects reflected light from the user's wrist.

Hemoglobin in blood absorbs green light. Thus, the amount of light absorbed at the wrist changes as the volume of the blood vessel changes. Accordingly, as illustrated in FIG. 4D, the amount of reflected light, which is light that has been emitted from the light-emitter unit 13a and then has been reflected at the wrist, changes depending on the volume of the blood vessel. Therefore, the volume pulse wave at the user's wrist is successfully detected from a change in the amount of light detected by the light-detector unit 13b.

### Display

The display 15 is, for example, a liquid crystal display (LED) or an organic electroluminescent (EL) (organic light-emitting diode (OLED)) display. The display 15 is disposed on the outer surface of the casing 10. The display 15 includes a display surface and displays an image (mirror image) of the user captured by the camera 11 in real time as illustrated in FIG. 4A, for example. The display 15 may display biological information including the user's blood pressure measured by the blood pressure measurement device 100. The display 15 may display other information (for example, the time, the date, and so forth).

### Processing Circuit

The processing circuit 16 is included in the casing 10 and includes a processor, a memory, etc. The processing circuit 16 performs a blood pressure measurement process. Specifically, the processing circuit 16 calculates a first pulse wave timing from a temporal change in luminance value in the user's cheek region in a plurality of images captured by an image capturing unit 101. The processing circuit 16 further determines a second pulse wave timing from a pulse wave detected by the pulse wave sensor 13 at the user's wrist. The processing circuit 16 then estimates the user's blood pressure from a time difference between the first pulse wave timing and the second pulse wave timing. Details about the process performed by the processing circuit 16 will be described later with reference to the drawings.

### Band

The band 17 is wound around the user's wrist. That is, the band 17 is a belt-like member that is wound entirely or partially around the user's wrist. The band 17 is, for example, a strap or bracelet formed of a resin, a metal, or a fiber. The band 17 may be integrally formed with the casing 10 or may be removable from the casing 10.

### Functional Configuration of Blood Pressure Measurement Device

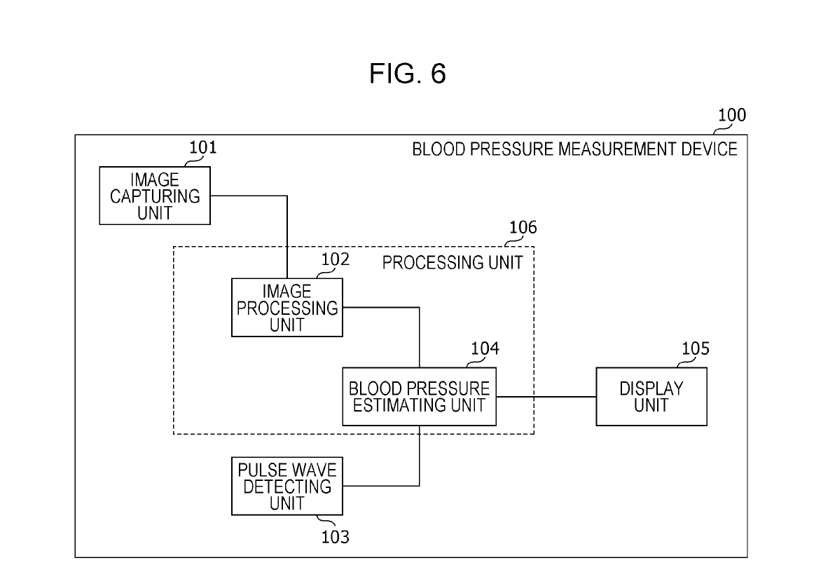

A functional configuration of the blood pressure measurement device 100 will be described next. FIG. 6 is a block diagram illustrating the functional configuration of the blood pressure measurement device 100 according to the first embodiment. As illustrated in FIG. 6, the blood pressure measurement device 100 includes the image capturing unit 101, a pulse wave detecting unit 103, a display unit 105, and a processing unit 106 in terms of its functions.

The image capturing unit 101 is implemented by, for example, the camera 11. The image capturing unit 101 continuously captures a plurality of images in terms of time. The plurality of captured images are sent to the processing unit 106. The image capturing unit 101 may send the plurality of images to the processing unit 106 after associating the time (i.e., image capturing time) at which each of the plurality of images has been captured with the image.

The pulse wave detecting unit 103 is implemented by, for example, the pulse wave sensor 13. The pulse wave detecting unit 103 detects a pulse wave at the user's wrist. Information on the pulse wave detected at the user's wrist is sent to the processing unit 106.

The pulse wave detecting unit 103 constantly detects the pulse wave and determines the peak. This configuration consequently allows the display unit 105 to constantly display vital data, such as pulse rate.

Note that the pulse wave detecting unit 103 need not necessarily constantly detect a pulse wave. For example, the pulse wave detecting unit 103 may start detection of a pulse wave when an image processing unit 102 succeeds in recognition of the eyes, ears, and nose in an image. This configuration successfully reduces energy consumption at a battery or the like.

The display unit 105 is implemented by, for example, the display 15. The display unit 105 displays various kinds of information. Specifically, the display unit 105 displays a mirror image of the user captured by, for example, the image capturing unit 101 in real time. The display unit 105 also displays the measured blood pressure of the user.

The processing unit 106 is implemented by, for example, the processing circuit 16. The processing unit 106 performs a process for measuring blood pressure. As illustrated in FIG. 6, the processing unit 106 includes the image processing unit 102 and a blood pressure estimating unit 104.

The image processing unit 102 receives, from the image capturing unit 101, a plurality of images of the user's face and the image capturing times attached to the respective images and calculates a first pulse wave timing from the plurality of images. A pulse wave timing is a time point of a feature point in the waveform of the pulse wave. For example, a pulse wave timing is a time point of a peak in the waveform of the pulse wave.

The blood pressure estimating unit 104 estimates the user's blood pressure from a time difference between the first pulse wave timing and a second pulse wave timing that is determined from the user's pulse wave detected by the pulse wave detecting unit 103.

### Operation of Blood Pressure Measurement Device

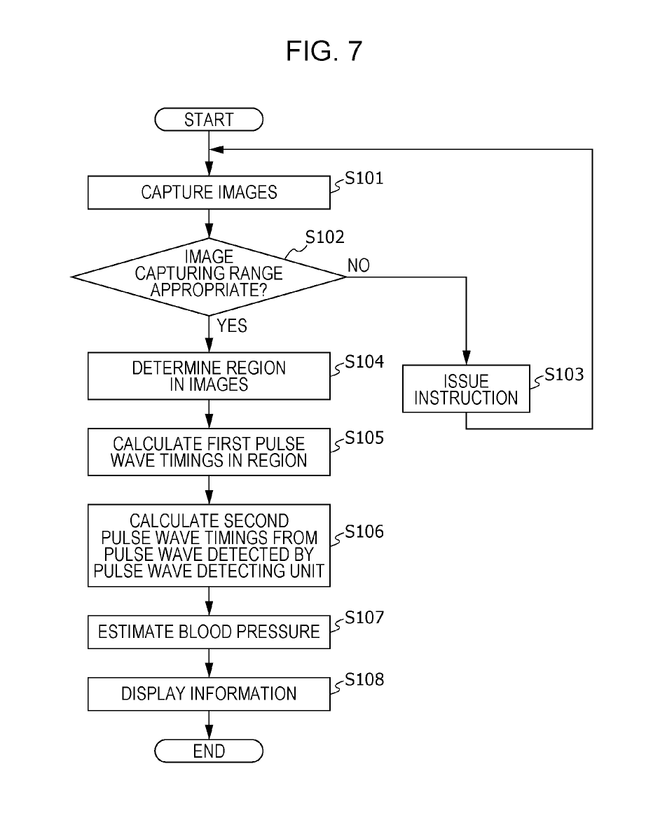

An operation performed by the blood pressure measurement device 100 thus configured will be described next. FIG. 7 is a flowchart illustrating a process performed by the blood pressure measurement device 100 according to the first embodiment.

First, the image capturing unit 101 captures a plurality of images of the user over time (step S101). For example, the image capturing unit 101 starts capturing images in response to a user operation on the blood pressure measurement device 100.

Then, the image processing unit 102 determines whether the image capturing region (range) is appropriate (step S102). Specifically, the image processing unit 102 determines whether, for example, the user's cheek region is successfully located in the plurality of images of the user. For example, the image processing unit 102 may recognize the user's features (the eyes, the ears, and the nose, for example) in the images and may determine whether the image capturing region is appropriate on the basis of the recognition result. Any recognition method may be used to recognize features. For example, features may be recognized in the images through pattern matching using pre-stored images of features.

For example, the image processing unit 102 calculates the relative position of the camera 11 with respect to the user's face on the basis of the plurality of images. Specifically, the image processing unit 102 calculates the distance L from the sizes of the features recognized in the images with reference to data stored in a memory (not illustrated), for example. The image processing unit 102 then determines that the image capturing region is appropriate if the calculated distance L is in a predetermined range. The predetermined range may be determined empirically or experimentally and may be, for example, greater than or equal to 10 cm and less than or equal to 40 cm. The memory is just required to store data indicating a relationship between the sizes of the features and the distance L between the blood pressure measurement device 100 and the user's cheek.

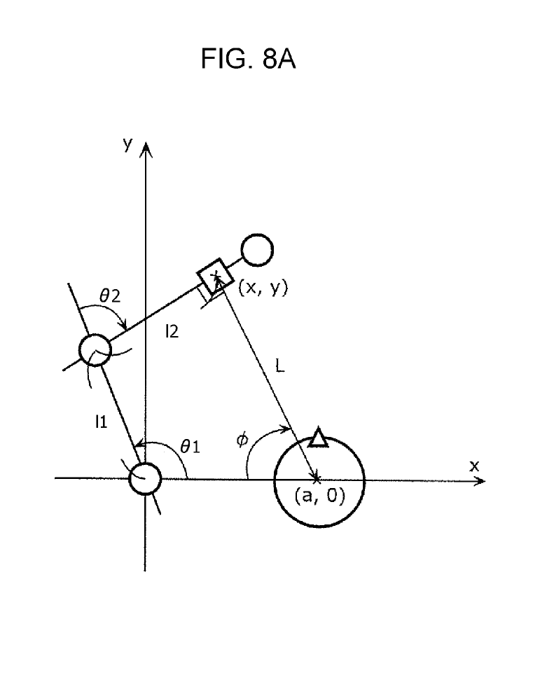

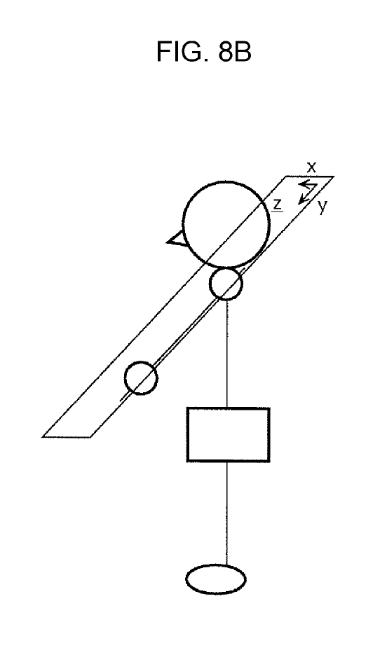

If it is determined that the image capturing region is inappropriate (NO in step S102), the processing unit 106 issues an instruction for adjusting the positional relationship between the user's face and the camera 11 (step S103). The process then returns to step S101. That is, the processing unit 106 causes the display unit 105 to display an instruction for changing the positional relationship between the user's face and the camera 11 on the basis of the relative position of the user's face with respect to the camera 11. For example, the processing unit 106 causes the display unit 105 to display an instruction, such as “Please bend your shoulder/elbow” or “Please twist your wrist”, on the basis of biomechanics models illustrated in FIGS. 8A and 8B. Referring to FIG. 8A, l1 denotes the length from the user's shoulder to the user's elbow, l2 denotes the length from the user's elbow to the blood pressure measurement device 100, and θ1 and θ2 respectively denote an angle of the shoulder joint and an angle of the elbow joint.

In addition, as illustrated in FIG. 8B, a plane formed by connecting three points, which are the shoulders and the elbow, is defined as a work plane z. Since the lengths l1 and l2 are constant, the distance L is determined depending on the angle of the shoulder joint θ1 and the angle of the elbow joint θ2. Accordingly, if the distance L derived from the sizes of the features (the eyes, the ears, and the nose, for example) recognized in the captured images is not in the predetermined range, the processing unit 106 gives an instruction for adjusting the angles of the shoulder joint and elbow joint θ1 and θ2 to the user.

Since the variable range of the angle of the elbow joint θ2 is larger than the variable range of the angle of the shoulder joint θ1, the user may be instructed to adjust only the angle of the elbow joint θ2 (to bend or stretch the elbow) without adjusting the angle of the shoulder joint θ1. For example, if the distance L derived from the images is smaller than the lower limit of the predetermined range, the processing unit 106 instructs the user to “stretch their elbow”, that is, to make the angle of the elbow joint θ2 smaller.

Specifically, the processing unit 106 calculates the relative position of the user's face with respect to the camera 11 on the basis of the size and position of at least one of the user's eyes, ears, and nose in the plurality of images of the user. The relative position is represented by the distance L between the camera 11 and the user's face and a positional shift of the user's face in the left-right and top-bottom directions.

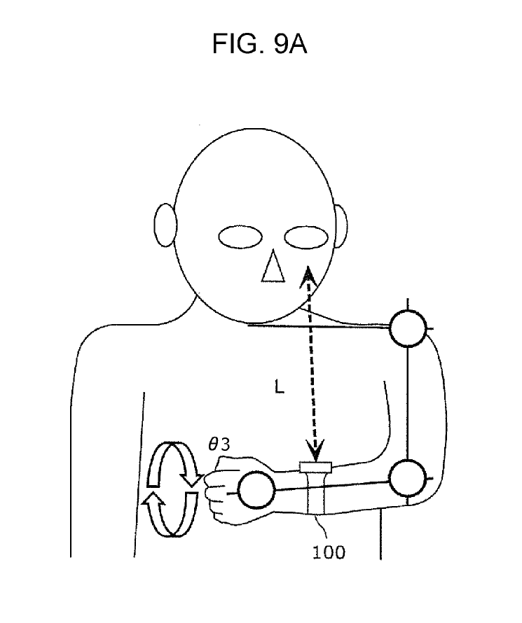

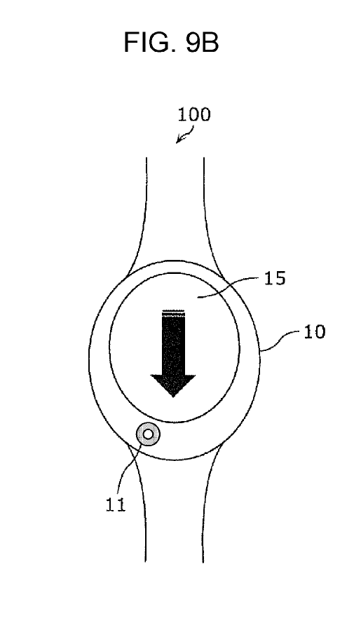

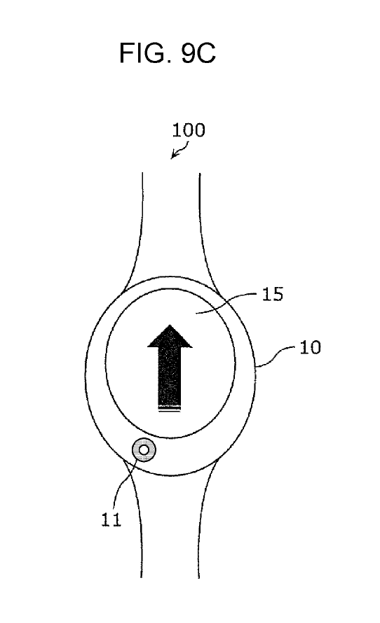

For example, even when the distance L derived from the images is in the predetermined range, the processing unit 106 instructs the user to adjust a rotation angle θ3 in a direction in which the wrist is twisted (i.e., instructs the user to twist their wrist) as illustrated in FIG. 9A if the position of the user's face is shifted from the center in the top-bottom direction in the images. For example, if the user's eyes, ears, nose are in an upper portion of the images, the processing unit 106 displays an instruction for twisting the wrist inward as illustrated in FIG. 9B. Conversely, if the user's eyes, ears, and nose are in a lower portion of the images, the processing unit 106 displays an instruction for twisting the wrist outward as illustrated in FIG. 9C.

In addition, if the position of the user's face is shifted from the center in the left-right direction in the images, the processing unit 106 gives the user an instruction for adjusting the angle of the shoulder joint θ1 so as to control the relative position of the user's face with respect to the camera 11.

If it is determined that the image capturing region is appropriate (YES in step S102), the image processing unit 102 determines a region in which a pulse wave is detected in the images (step S104). Specifically, the image processing unit 102 determines a region used to measure a pulse wave described below.

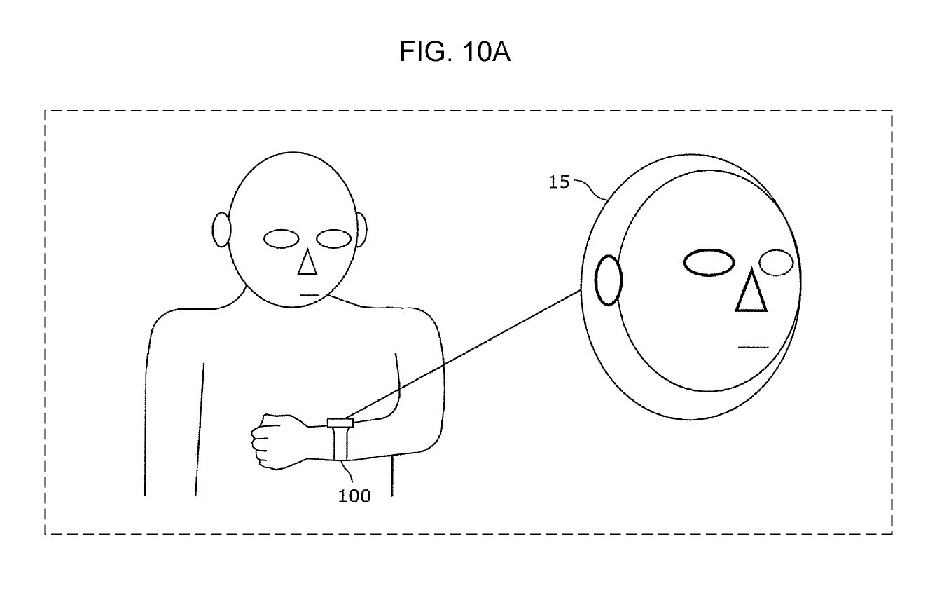

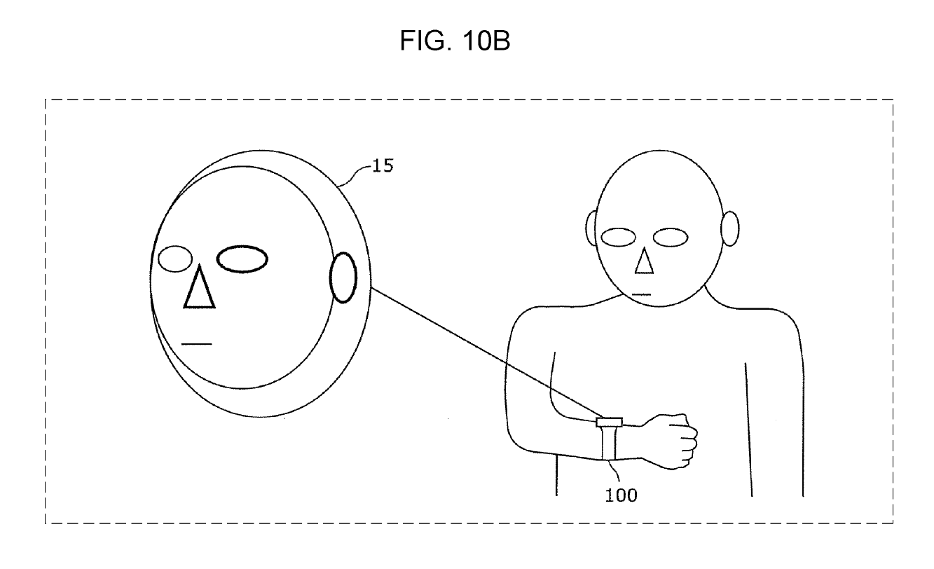

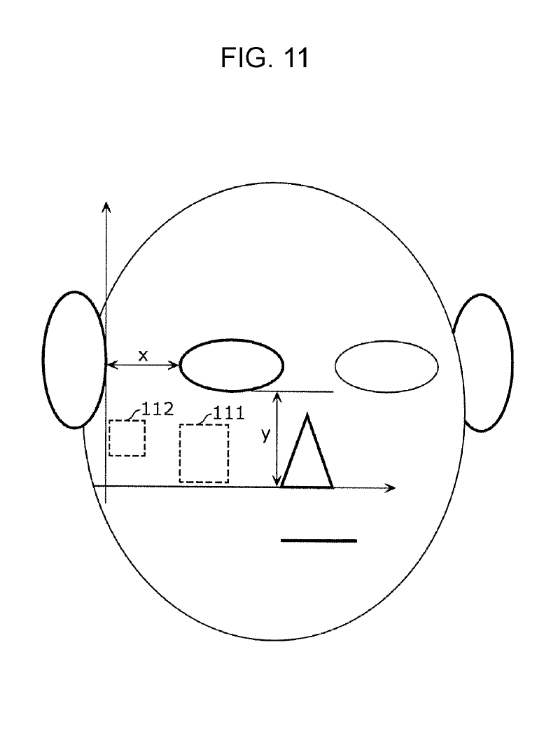

FIGS. 10A and 10B are diagrams for describing a method used by the image processing unit 102 to determine the region in the images. For example, in the case where the blood pressure measurement device 100 is worn on the left wrist as illustrated in FIG. 10A, the image capturing unit 101 captures images of the left side of the user's face. Thus, resultant images include the user's left eye, left ear, and nose. In addition, in the case where the blood pressure measurement device 100 is worn on the right wrist as illustrated in FIG. 10B, resultant images include the user's right eye, right ear, and nose.

Thus, the image processing unit 102 recognizes one of the eyes, one of the ears, and the nose in the images and determines a region in which a pulse wave is detected on the basis of the recognition result.

FIG. 11 illustrates a method for determining the region in the images on the basis of one of the eyes, one of the ears, and the noise. Let x denote the distance between the recognized ear and the recognized eye and y denote the distance between the recognized eye and the recognized nose, and the origin is defined by the inner-side end of the ear and the lower end of the nose. Then, the image processing unit 102 determines coordinates of the left upper corner of a region 111 for detecting a pulse wave to be (x, 2y/3), determines the width of the region 111 to be 2x/3, and determines the height of the region 111 to be 2y/3.

Note that the position of the pulse wave detection region is not limited to this example. Since a region suitable for detection of a pulse wave varies depending on the user, lighting, or the like, the region may be determined in accordance with the user or the environment. In addition, the image processing unit 102 may determine a plurality of regions (regions 111 and 112) as illustrated in FIG. 11.

The image processing unit 102 calculates a first pulse wave timing from luminance values in the region determined in step S104 (step S105). Blood is sent out from the heart to body parts, such as the face and hands, as a result of contraction of the heart. At that time, as the heart contracts, the blood vessel pulses, and consequently the volume of the blood vessel periodically changes.

Luminance at the face or hand in captured images changes depending on the amount of hemoglobin or the like in blood. In images captured under visible light, luminance changes greatly in a frequency band near frequencies for green light. For example, luminance for green (G) at the face obtained when lots of blood is at the face (that is, the volume of the blood vessel is large) is smaller than luminance for green (G) at the face obtained when less blood is at the face (that is, the volume of the blood vessel is small).

The image processing unit 102 calculates the first pulse wave timing by using this temporal change in luminance. A pulse wave timing is a time point of a feature point in the waveform of the pulse wave. An example of the feature point is a peak in the waveform. For example, when an image Xi denotes an image captured at a time point ti and luminance li denotes luminance obtained from the image Xi (1≦l≦n, and i and n are natural numbers), the “temporal change in luminance” may be considered to be a set of (ti, li) values. The waveform indicating the temporal change in luminance may be considered to be a waveform that is derived by plotting the (ti, li) values in the coordinate system in which the horizontal axis represents time and the vertical axis represents luminance.

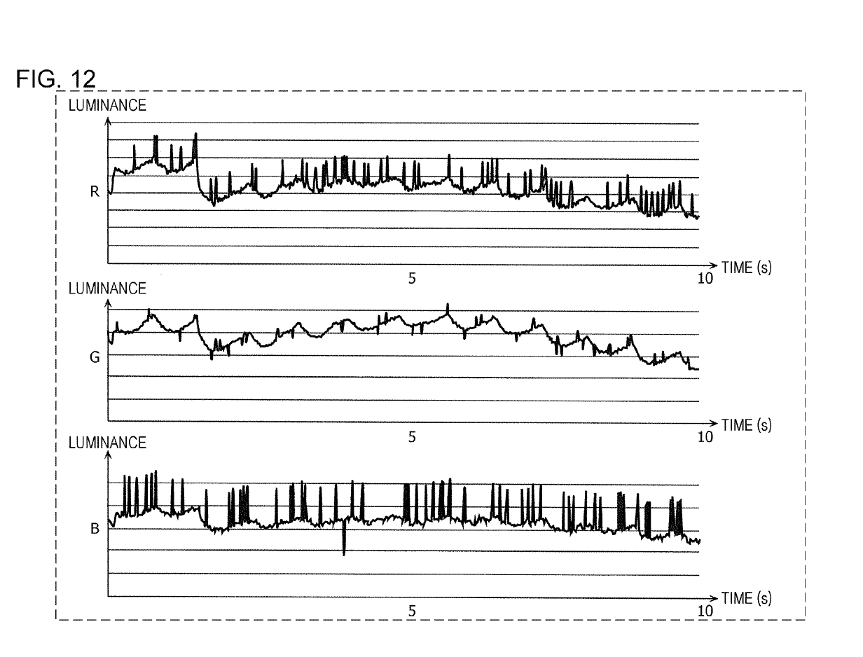

FIG. 12 illustrates temporal changes in luminance for red (R), green (G), and blue (B) in the cheek region in images of the face captured by the image capturing unit 101. Referring to FIG. 12, the horizontal axis represents time, and the vertical axis represents luminance. As FIG. 12 indicates, luminance for R, luminance for G, and luminance for B change periodically due to a pulse wave. Since images contain noise, such as scattered light, signal processing, such as filtering, may be applied to the image signals to remove the noise. In the first embodiment, the image processing unit 102 calculates a timing of each peak in the waveform of the pulse wave as a first pulse wave timing, by using lowpass-filtered luminance for G. For example, the image processing unit 102 detects a peak in the waveform of the pulse wave by using the hill climbing or local search, for example.

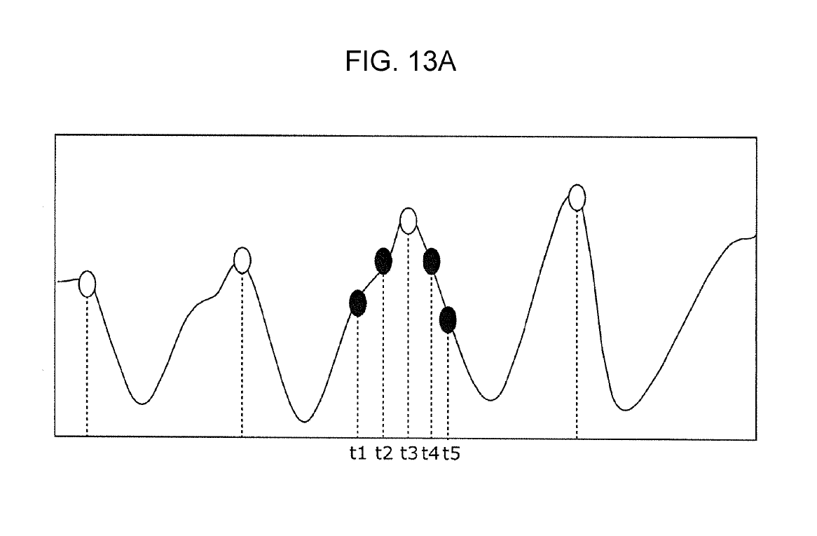

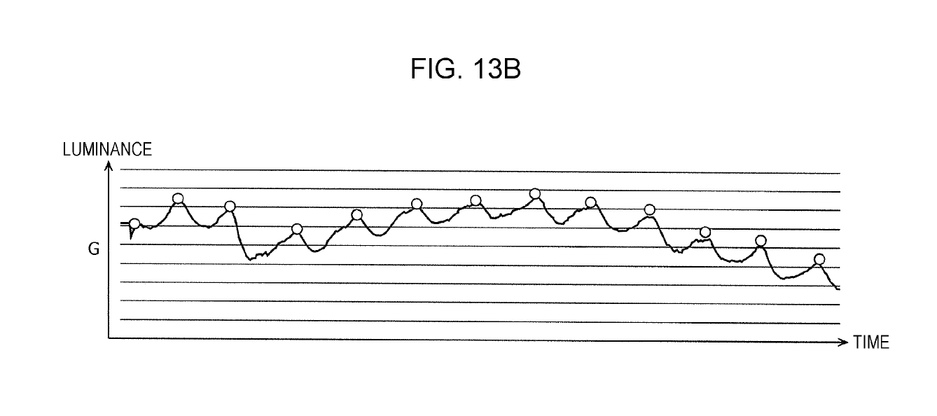

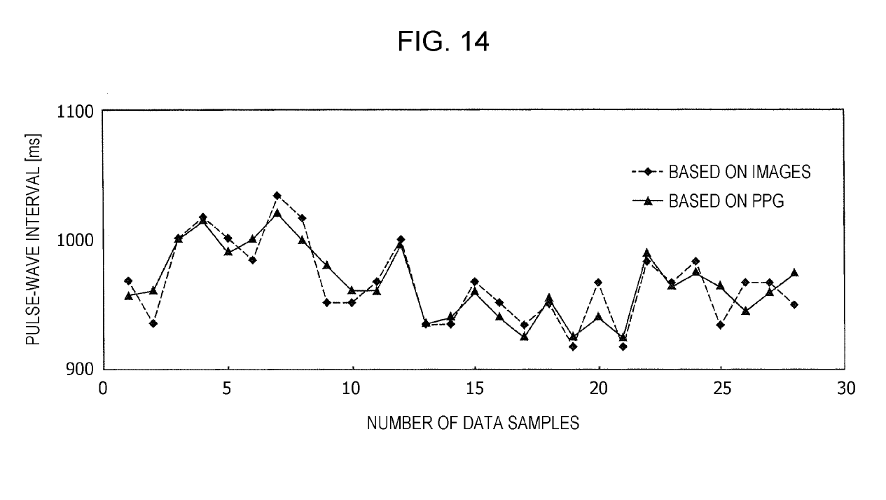

FIGS. 13A and 13B are diagrams for describing how a peak is detected in the waveform of a pulse wave. Referring to FIG. 13A, suppose that processing is performed sequentially in chronological order for a plurality of time points on the waveform and that a time point t2 is the current processing-target time point. In this case, the image processing unit 102 compares the luminance value at the current processing-target time point t2 with the luminance value at an immediately preceding time point t1. Likewise, the image processing unit 102 compares the luminance value at the current processing-target time point t2 with the luminance value at an immediately following time point t3. In FIG. 13A, the luminance value at the time point t2 is larger than the luminance value at the time point t1 but is smaller than the luminance value at the time point t3. Thus, the image processing unit 102 determines that the luminance value at the current processing-target time point t2 is not a peak and increments the processing-target time point by 1. As a result, the time point t3 is set as the current processing-target time point. The luminance value at the time point t3 is larger than the luminance value at the immediately preceding time point t2 and than the luminance value at the immediately following time point t4. Thus, the image processing unit 102 determines that the luminance value at the time point t3 is a peak and derives the time point t3 as the first pulse wave timing. FIG. 13B is a graph of lowpass-filtered luminance for G. Referring to FIG. 13B, each circle represents luminance at the first pulse wave timing calculated through the peak search.

FIG. 14 is a diagram for describing the fact that pulse wave timings calculated from the images correlate to the actual pulse wave. In FIG. 14, the solid line represents pulse wave timings calculated from a pulse wave measured at the fingertip by a fingertip photoplethysmography sensor. In addition, a dash line represents pulse wave timings calculated from images of the face captured simultaneously with measurement of the pulse wave at the fingertip. The vertical axis represents the time interval (ms) between pulse wave timings. FIG. 14 indicates that the time interval between pulse wave timings is not constant and varies in a range from approximately 920 ms to 1050 ms in FIG. 14. FIG. 14 also indicates that pulse wave timings of the pulse wave measured at the fingertip by a fingertip photoplethysmography sensor and pulse wave timings calculated from the images have a very high time correlation. This fact indicates that the number of pulses per minute and pulse wave timings based on the waveform of a pulse wave can be detected relatively accurately from a change in luminance in images.

The blood pressure estimating unit 104 calculates a second pulse wave timing from the pulse wave detected at the wrist by the pulse wave detecting unit 103 (step S106). The second pulse wave timing corresponds to the first pulse wave timing. Note that a method for associating the first pulse wave timing and the second pulse wave timing will be described later. Note that a method for calculating the second pulse wave timing may be substantially the same as the method for calculating the first pulse wave timing. That is, the second pulse wave timing may be calculated from the feature point in the waveform. An example of the feature point is a peak in the waveform. The method for detecting a peak in the waveform may be substantially the same as the method described with reference to FIGS. 13A and 13B.

The blood pressure estimating unit 104 estimates blood pressure on the basis of a time difference between the first pulse wave timing calculated in step S105 and the second pulse wave timing calculated in step S106 (step S107). This time difference occurs because time taken for the pulse wave to propagate from the heart differs and is called a differential pulse transit time.

In general, it is considered that the time from when the heart contracts to when blood reaches a fingertip or the like from the heart (pulse transit time) and blood pressure have a correlation. The higher the blood pressure, the shorter the pulse transit time; the lower the blood pressure, the longer the pulse transit time. In addition, methods for estimating blood pressure by representing these relationships using a predetermined approximate expression are known. In the first embodiment, the blood pressure estimating unit 104 estimates blood pressure in accordance with (Equation 2) below, for example.

P=αt+β (Equation 2)

In (Equation 2), t denotes the differential pulse transmit time and α and β represent coefficients. In the first embodiment, for example, coefficients of α=−1.5 and β=185 are used.

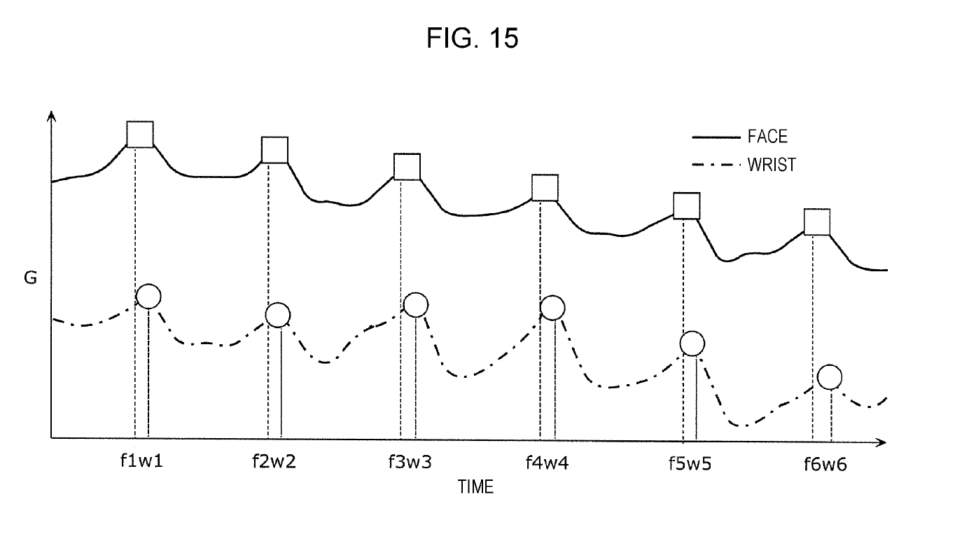

FIG. 15 is a diagram for describing how blood pressure is estimated. Referring to FIG. 15, the vertical axis represents luminance in face images and luminous intensity at a photoplethysmography sensor worn on the wrist, and the horizontal axis represents time.

Each square represents the first pulse wave timing calculated from the change in luminance in the face images. Time points of the first pulse wave timings are f1, f2, f3, f4, f5, and f6 in chronological order.

Each circle represents the second pulse wave timing calculated from the change in luminous intensity at the wrist. Time points of the second pulse wave timings are w1, w2, w3, w4, w5, and w6 in chronological order.

There is a certain time difference between the first pulse wave timing and the second pulse wave timing. The blood pressure estimating unit 104 estimates blood pressure on the basis of this time difference.

The blood pressure estimating unit 104 may associate the first pulse wave timings (e.g., f1, f2, f3, f4, f5, and f6) with the respective second pulse wave timings (e.g., w1, w2, w3, w4, w5, and w6). This association may be performed in accordance with a method described below.

The blood pressure estimating unit 104 acquires, from the image processing unit 102, the first pulse wave timings (e.g., f1, f2, f3, f4, f5, and f6) calculated by the image processing unit 102 in step S105. The blood pressure estimating unit 104 holds the second pulse wave timings (e.g., w1, w2, w3, w4, w5, and w6) calculated by the blood pressure estimating unit 104 in step S106. The blood pressure estimating unit 104 determines which of the second pulse wave timings w1, w2, w3, w4, w5, and w6 corresponds to, for example, the first pulse wave timing f3 among the first pulse wave timings f1, f2, f3, f4, f5, and f6 in a manner described below, for example. The blood pressure estimating unit 104 associates the second pulse wave timing w3 that is the closest to the first pulse wave timing f3 with the first pulse wave timing f3 from among the second pulse wave timings that follow the first pulse wave timing f3. The blood pressure estimating unit 104 also determines the second pulse wave timing that correspond to each of the first pulse wave timings other than the first pulse wave timing f3 by using a method similar to the above-described one. In the example illustrated in FIG. 15, the blood pressure estimating unit 104 determines that the timing f1 corresponds to the timing w1, the timing f2 corresponds to the timing w2, the timing f4 corresponds to the timing w4, the timing f5 corresponds to the timing w5, and the timing f6 corresponds to the timing w6.

For example, the time difference between the timing f1 and the timing w1 is 50 ms. Accordingly, the blood pressure estimating unit 104 estimates blood pressure to be 110 mmHg (=−1.5×50+185) by substituting this time difference to (Equation 2). Note that an average of a plurality of differential pulse transit times may be used as the differential pulse transit time.

For example, in FIG. 15, six measured differential pulse transit times are 50 ms (w1−f1), 45 ms (w2−f2), 53 ms (w3−f3), 47 ms (w4−f4), 52 ms (w5−f5), and 53 ms (w6−f6). Accordingly, the blood pressure estimating unit 104 may estimate blood pressure to be 110 mmHg by using 50 ms, which is the average. There are cases where pulse wave timings contain errors and where pulse wave timings are not measured accurately due to the influence of noise in the usual environment. The use of a plurality of differential pulse transmit times implements more robust blood pressure measurement.

The display unit 105 displays health-related information, such as the pulse wave obtained from the image processing unit 102 and the pulse wave detecting unit 103 and the blood pressure obtained from the blood pressure estimating unit 104 (step S108).

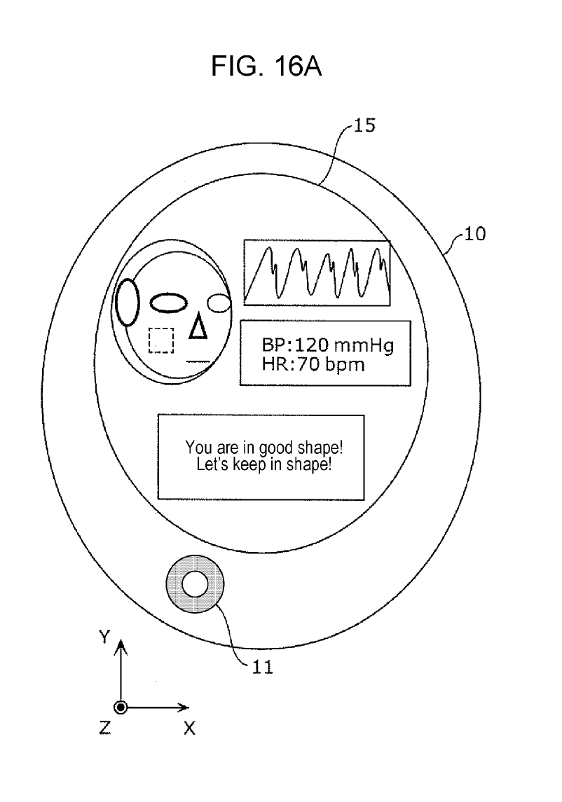

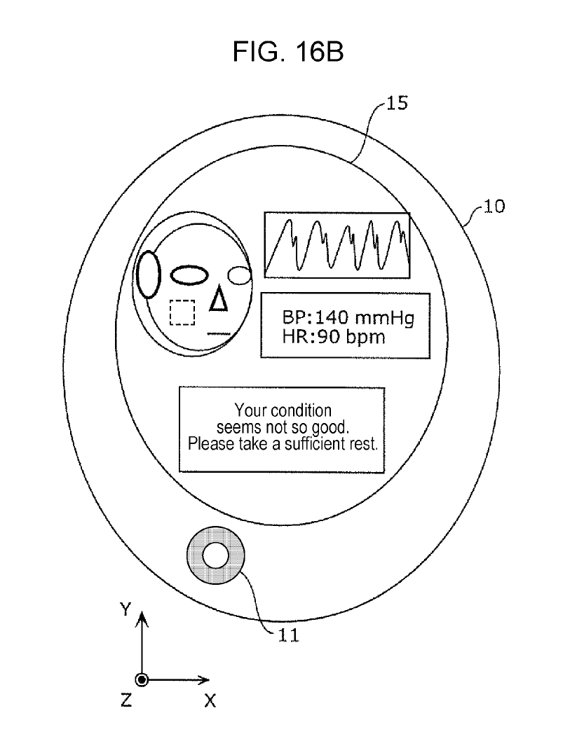

FIGS. 16A and 16B each illustrate information displayed on the display unit 105. The size of the face image that has been displayed on the display unit 105 in a large size is reduced as illustrated in FIG. 16A after blood pressure has been estimated. Also, the temporal change in luminance obtained from the image processing unit 102 (i.e., the waveform of the pulse wave), the blood pressure estimated by the blood pressure estimating unit 104, and the pulse rate (heart rate) calculated by the image processing unit 102 or the pulse wave detecting unit 103 are displayed.

FIG. 16A illustrates a relatively healthy state (BP: 120 mmHg, HR: 70 bpm), whereas FIG. 16B illustrates a relatively unhealthy state (BP: 140 mmHg, HR: 90 bpm). In the healthy state illustrated in FIG. 16A, an advice for staying in healthy is displayed. On the other hand, in the unhealthy state illustrated in FIG. 16B, an advice for recommending that the user take a rest is displayed since the user may be under a pressure or be tired and may feel stressed out.

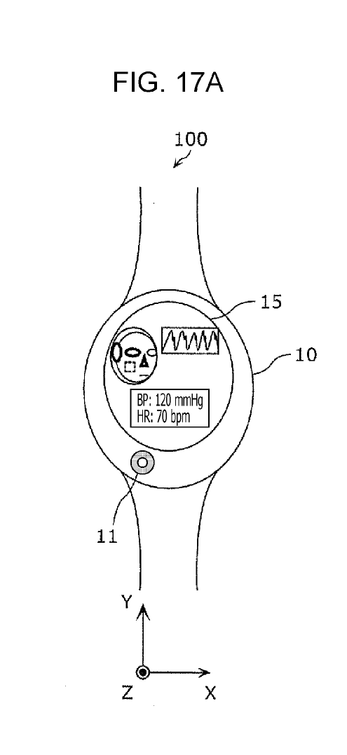

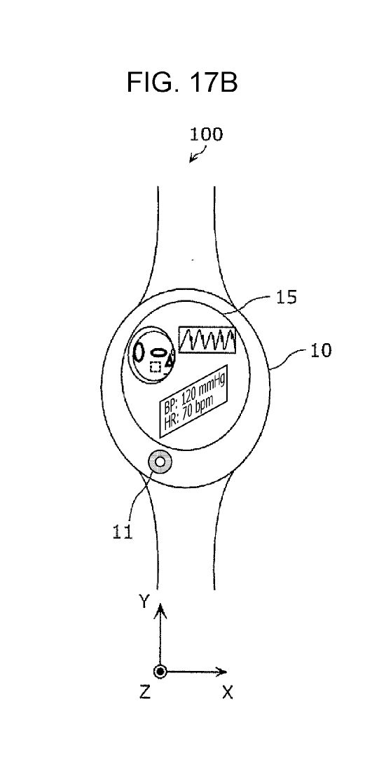

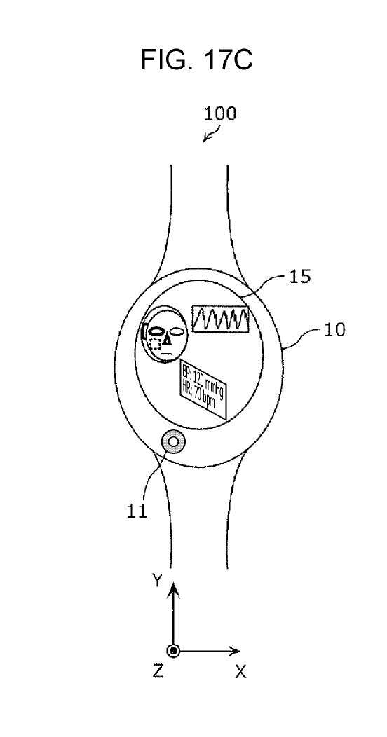

FIGS. 17A to 17C illustrate other examples of information displayed on the display unit 105. Specifically, FIG. 17A illustrates a display example in the case where the positional relationship between the camera 11 and the user's face is appropriate. FIG. 17B illustrates a display example in the case where the camera 11 is directed toward the right when viewed from the user. FIG. 17C illustrates a display example in the case where the camera 11 is directed toward the left when viewed from the user.

Referring to FIG. 17A, blood pressure and pulse rate are displayed on the display unit 105 horizontally since blood pressure can be measured highly accurately without changing the relative positional relationship between the user's face and the camera 11.

Referring to FIG. 17B, since the camera 11 is directed toward the right when viewed from the user, the image capturing region is shifted to the right side of the user. Accordingly, blood pressure and pulse rate are displayed on the display unit 105 so as to be tilted rightward when viewed from the user. In this case, the user moves their wrist such that the camera 11 is tilted leftward when viewed from the user or moves their face to the right relative to the camera 11 in order to see the displayed blood pressure and pulse rate. As a result, the positional relationship between the camera 11 and the user's face approaches the relationship illustrated in FIG. 17A, and the blood pressure measurement accuracy can be increased.

Referring to FIG. 17C, since the camera 11 is directed toward the left when viewed from the user, the image capturing region is shifted to the left side relative to the user, in contrast to FIG. 17B. Accordingly, blood pressure and pulse rate are displayed on the display unit 105 so as to be tilted leftward when viewed from the user. In this case, the user moves their wrist such that the camera 11 is tilted rightward when viewed from the user or moves their face to the left relative to the camera 11 in order to see the displayed blood pressure and pulse rate. As a result, the positional relationship between the camera 11 and the user's face approaches the relationship illustrated in FIG. 17A, and the blood pressure measurement accuracy can be increased.

The cases where the image capturing region is shifted to the left and to the right have been described herein. In the cases where the image capturing region is shifted upward or downward, information such as blood pressure and pulse rate may be displayed toward the top or the bottom on the display unit 105.

### Advantageous Effects

As described above, in the blood pressure measurement device 100 according to the first embodiment, the optical axis of the camera 11 is successfully tilted in the left-right direction with respect to the direction of the normal to the display surface. Thus, it becomes easier to capture images of the user's face region used to calculate the first pulse wave timing, and consequently the blood pressure estimation accuracy can be increased. Specifically, appropriate images can be captured as a result of a user's movement intended to see information displayed on the display 15, and blood pressure can be measured relatively easily.

In addition, with the blood pressure measurement device 100 according to the first embodiment, the first pulse wave timing can be calculated on the basis of a temporal change in luminance value in the cheek region, and consequently the blood pressure estimation accuracy can be increased.

Further, with the blood pressure measurement device 100 according to the first embodiment, an instruction for changing the positional relationship between the user's face and the camera 11 can be displayed on the display 15 on the basis of the relative position of the user's face when the user's cheek region is not successfully located in the images. Accordingly, the user can appropriately change the positional relationship between their face and the camera 11, and consequently images of the user's cheek region can be captured easily.

In addition, with the blood pressure measurement device 100 according to the first embodiment, the recognition models can be switched between in accordance with the position where the blood pressure measurement device 100 is worn. Accordingly, the relative position of the user's face can be calculated more accurately, and consequently a more appropriate instruction can be displayed.

In addition, with the blood pressure measurement device 100 according to the first embodiment, at least one of an instruction for twisting the wrist and an instruction for bending or stretching the elbow can be displayed on the display 15. Accordingly, the user can make a move in accordance with an intuitive and easy-to-understand instruction, and consequently it becomes easier to adjust the relative position of the camera 11.

### Second Embodiment

In a second embodiment, when the amount of light incident on the user's face is insufficient, an instruction for moving the position of a blood pressure measurement device is displayed on the basis of luminance of an image captured by an image capturing unit.

Since the structure and functional configuration of the blood pressure measurement device according to the second embodiment is substantially the same as or similar to those of the blood pressure measurement device according to the first embodiment, illustrations and descriptions thereof are omitted appropriately.

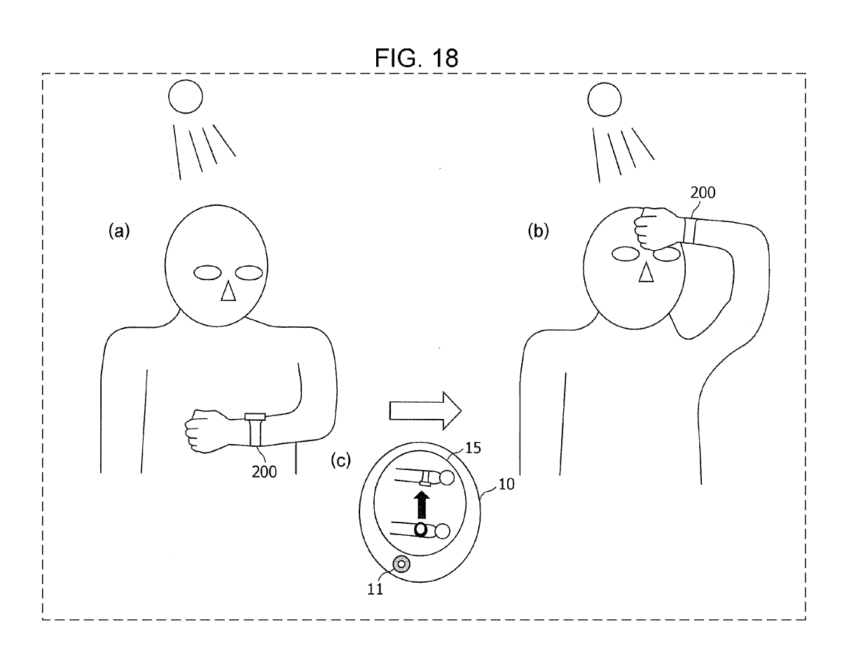

FIG. 18 is a diagram illustrating an example of how an instruction is given to the user by a blood pressure measurement device 200 according to the second embodiment.

The user is illuminated by light from a lighting apparatus indoors and is illuminated by sunlight outdoors. Accordingly, the light source is typically located above the user. When the user takes images of their cheek by using the blood pressure measurement device 200 in order to measure blood pressure, the user looks down at the camera 11 from above as illustrated in FIG. 18(a). Thus, the probability of the user being backlight is high.

The image processing unit 102 determines whether the user is backlit on the basis of luminance of a captured image.

For example, the image processing unit 102 determines whether the user is backlit on the basis of the R, G, B luminance values at the user's cheek, the luminance values at the background, and the difference in luminance value between the cheek and the background in an image.

For example, if the background luminance value is greater than or equal to a first luminance threshold (e.g., 230 within a luminance range from 0 to 255) and the G luminance value in the cheek image is less than or equal to a second luminance threshold (e.g., 120), the blood pressure measurement device 200 may give the user an instruction for lifting their arm on which the blood pressure measurement device 200 is worn so that the blood pressure measurement device 200 is diagonally above their face as illustrated in FIG. 18(c). If the user looks up to see the display 15, the amount of light that is incident on the cheek increases as illustrated in FIG. 18(b). At that time, since the orientation of the camera 11 deviates from the direction of the light source, the luminance of the entire image decreases (for example, the average luminance for R, G, and B decreases to 200). On the other hand, since the orientation of the user's face becomes closer to the direction of the light source, the G luminance value in the cheek image increases (to 120-240, for example). As a result, the accuracy of the first pulse wave timing calculated from cheek images can be increased.

Note that the first luminance threshold and the second luminance threshold are not necessarily constant values. For example, the first luminance threshold may be determined on the basis of the average of the R, G, and B luminance values of an image captured with the camera 11 directed toward the light source. In addition, the second luminance threshold may be determined on the basis of the average luminance value for the entire face.

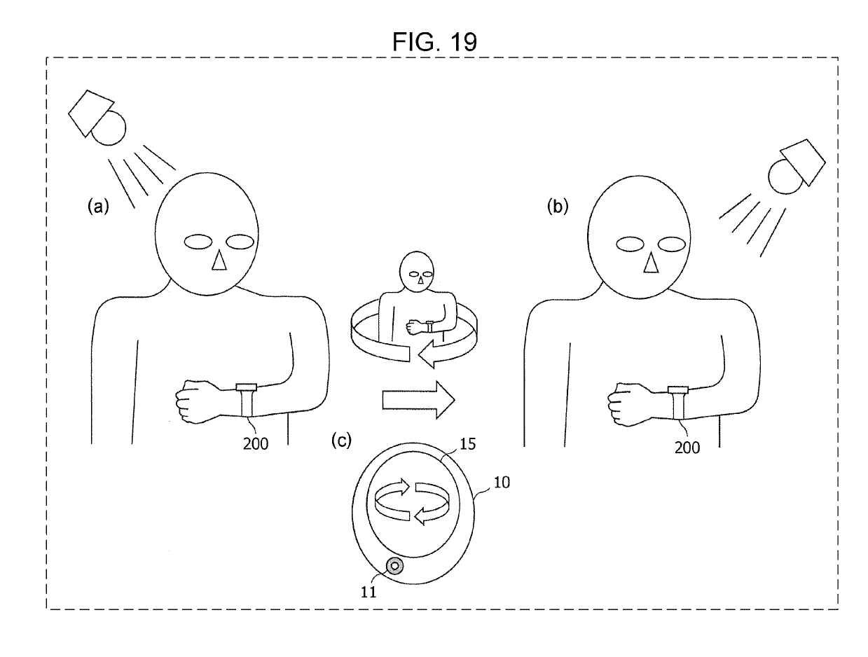

Further, if the luminance value at the cheek in the image is less than a third luminance threshold (e.g., 180) even after an instruction for lifting the arm is given to the user by the blood pressure measurement device 200, the issue regarding backlighting is not fully resolved as illustrated in FIG. 19(a). Accordingly, the blood pressure measurement device 200 may display on the display 15 an instruction for twisting the body as illustrated in FIG. 19(c). As a result of the user twisting their body in accordance with the instruction, the amount of light that is incident on the cheek increases as illustrated in FIG. 19(b), and consequently the accuracy of the pulse wave timing can be increased.

As described above, with the blood pressure measurement device 200 according to the second embodiment, an instruction for moving the blood pressure measurement device 100 to an upper position can be displayed on the display 15 if the user is backlit. Accordingly, the issue regrading backlighting can be resolved when the light source is located above the user, and consequently images more suitable for blood pressure estimation can be captured.

In addition, with the blood pressure measurement device 200 according to the second embodiment, an instruction for twisting the user's body can be displayed on the display 15 when the user is backlit. Accordingly, the issue regarding backlighting can be resolved when the light source is located on the side of the user, and consequently images more suitable for blood pressure estimation can be captured.

### Third Embodiment

Since the pulse transmit time changes due to influences of the gravity or the like when the user lifts their wrist in order to ensure a certain amount of light that is incident on their face as in the second embodiment, the blood pressure estimation accuracy may decrease in some cases. Accordingly, in a third embodiment, content of an instruction given to cope with the issue regarding backlighting is changed in accordance with the position (height) of the wrist.

Since the structure and functional configuration of the blood pressure measurement device according to the third embodiment is substantially the same as or similar to those of the blood pressure measurement device according to the first embodiment, illustrations and descriptions thereof are omitted appropriately.

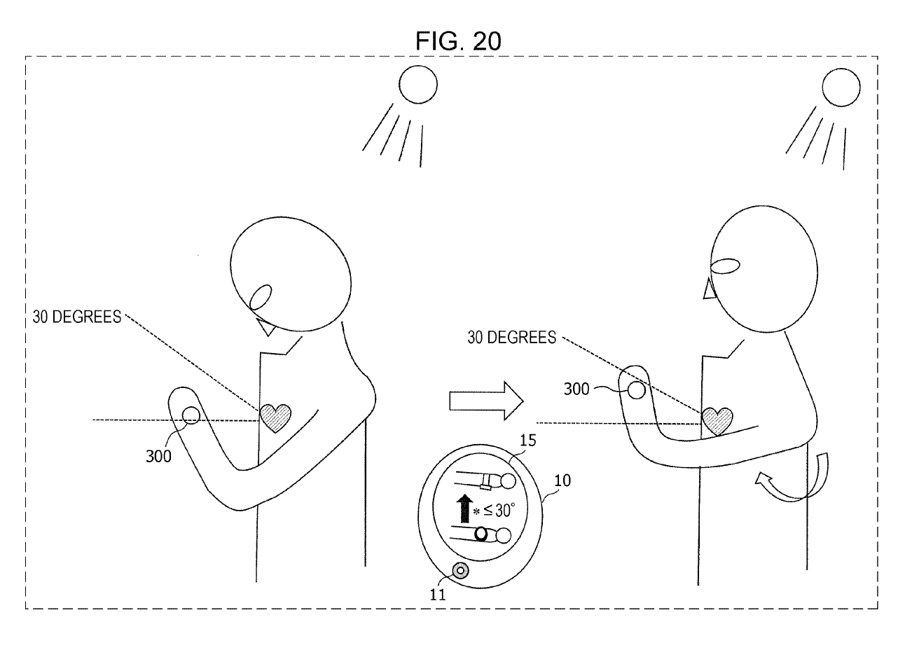

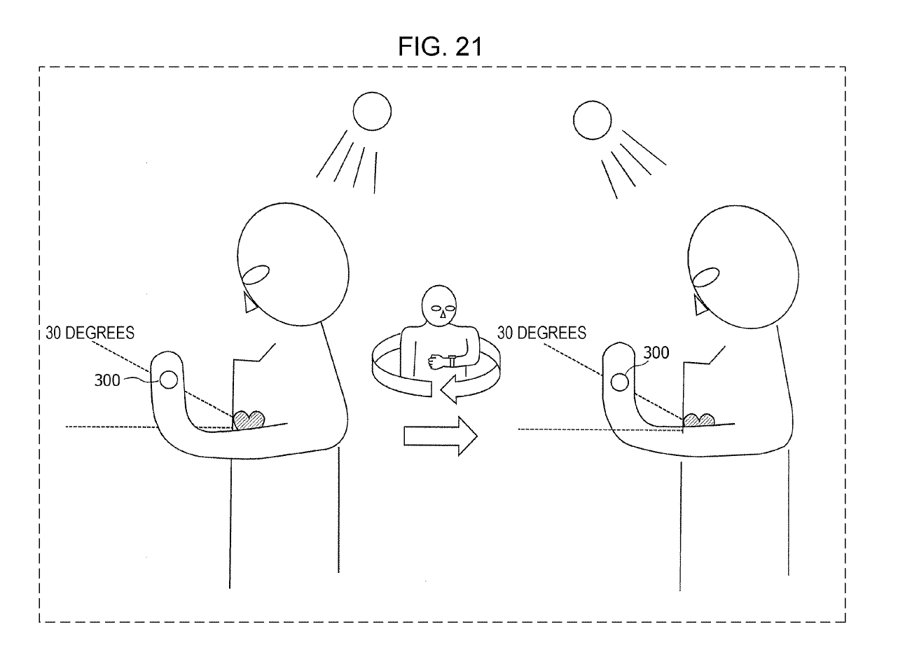

FIGS. 20 and 21 are diagrams each illustrating an example of how an instruction is given to the user by a blood pressure measurement device 300 according to the third embodiment. As illustrated in FIG. 20, a threshold height (e.g., 30 degrees) indicating the height of the wrist relative to the height of the heart is set in advance. If it is determined that the user is backlit as in the second embodiment, the blood pressure measurement device 300 estimates the height of the wrist. Specifically, the processing unit 106 estimates the height of the wrist on the basis of the size of the face in the image. If the value indicating the estimated height of the wrist is less than a threshold height, the blood pressure measurement device 300 displays an instruction for lifting the wrist in a range not exceeding the threshold height as illustrated in FIG. 20. In FIG. 20, an instruction for lifting the wrist in a range in which an angle between the horizontal plane relative to the position of the user's heart and the position of the blood pressure measurement device 300 does not exceed 30 degrees is displayed.

On the other hand, if the value indicating the height of the wrist is already equal to or greater than the threshold height as illustrated in FIG. 21, the blood pressure measurement device 300 displays an instruction for twisting the body. As described above, with the blood pressure measurement device 300 according to the third embodiment, an instruction can be given to the user in accordance with the posture of the user, and consequently blood pressure estimation can be performed accurately.

### Other Embodiments

While the blood pressure measurement devices according to one or a plurality of aspects of the present disclosure have been described above on the basis of the embodiments, the present disclosure is not limited to these embodiments. Embodiments achieved by applying various modifications conceived by a person skilled in the art to the embodiments and embodiments achieved by using elements of different embodiments in combination may also be within the scope of the one or plurality of aspects of the present disclosure as long as these embodiments do not depart from the essence of the present disclosure.

In addition, in the embodiments described above, the case where the blood pressure measurement device is worn on the back-of-hand side of the user's left wrist has been described as an example; however, the position where the blood pressure measurement device is worn is not limited to this example. For example, the blood pressure measurement device may be worn on the user's right wrist or on the palm side of the wrist. In this case, recognition models used to recognize features of the user in images may be switched between in accordance with the position where the blood pressure measurement device is worn.

For example, when the blood pressure measurement device is worn on the left wrist, images of the left side of the user's face are captured. Thus, the blood pressure measurement device recognizes the left eye, the left ear, and the nose as features. In addition, when the blood pressure measurement device is worn on the back-of-hand side of the wrist, the user twists their wrist inward to capture images of their face. As a result, images of the user's face are captured from the upper portion to the lower portion of the face. That is, images are captured in the order of the forehead, the cheek, and the chin. Accordingly, the blood pressure measurement device can calculate the first pulse wave timing in images of the cheek that are captured after a predetermined time has passed from a timing at which an image of the forehead is captured.

In addition, for example, when the blood pressure measurement device is worn on the right wrist, images of the right side of the user's face are captured. Thus, the blood pressure measurement device recognizes the right eye, the right ear, and the nose as features. In addition, when the blood pressure measurement device is worn on the palm side of the wrist, the user twists their wrist outward to capture images of their face. As a result, images of the user's face are captured from the lower portion to the upper portion of the face. That is, images are captured in the order of the chin, the cheek, and the forehead. Accordingly, the blood pressure measurement device can calculate the first pulse wave timing in images of the cheek that are captured after a predetermined time has passed from a timing at which an image of the chin is captured.

Which of the right wrist and the left wrist the user is wearing the blood pressure measurement device on may be determined on the basis of a user input. In addition, it may be determined automatically from captured images. For example, it can be determined that the blood pressure measurement device is worn on the left wrist when the captured images include the left ear, the left eye, and the nose. This configuration successfully reduces a calculation amount, compared with that of typical techniques for recognizing the entire face.

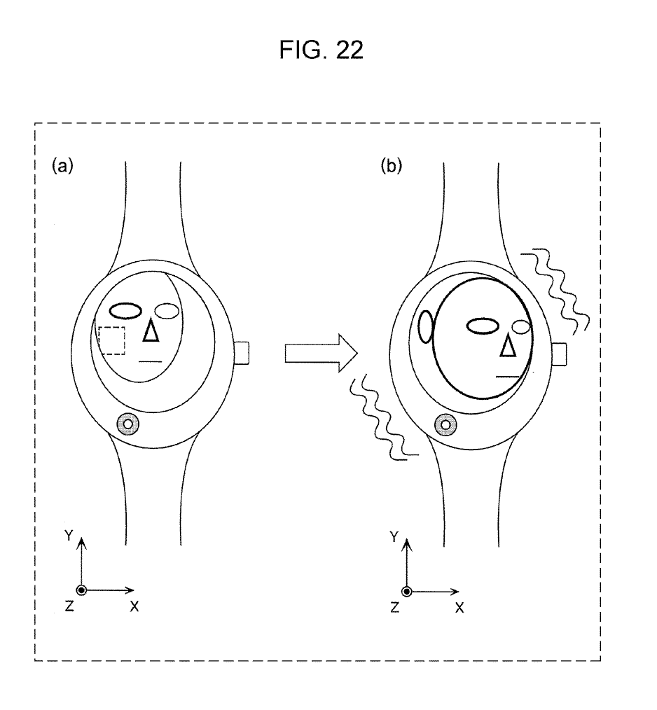

If it is determined that the image capturing range is appropriate in the embodiments described above, the user may be notified of the determination result through vibration of the blood pressure measurement device. For example, the blood pressure measurement device may vibrate its casing as illustrated in FIG. 22(b) upon recognizing features of the user (the left ear, the left eye, and the nose in this example) as illustrated in FIG. 22(a).

The blood pressure measurement device searches for peaks in the waveform of a pulse wave by using the hill climbing in the embodiments described above; however, the method used is not limited to this one. For example, the blood pressure measurement device may search for peaks in the waveform of a pulse wave by using autocorrelation or a differential function. That is, the blood pressure measurement device can use any search method as long as peaks in the waveform of a pulse wave are successfully retrieved.

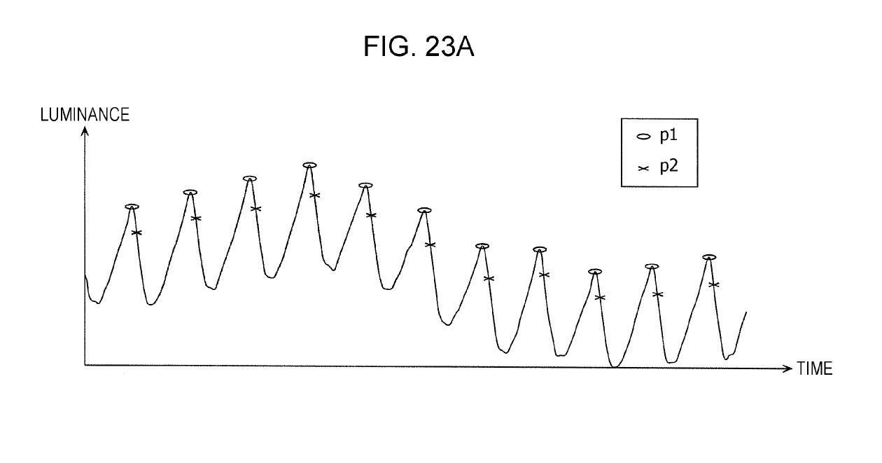

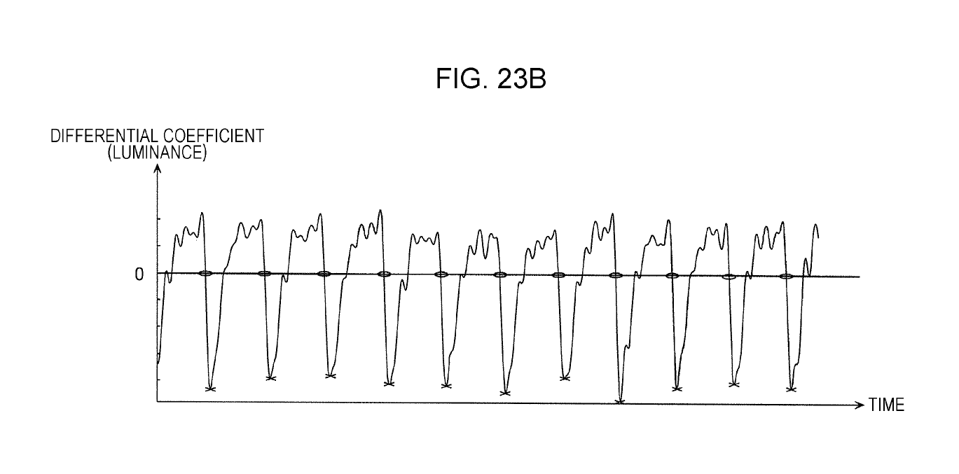

Pulse wave timings are calculated on the basis of peaks in the waveform of a pulse wave in the embodiments described above; however, the feature points to be used are not limited to the peaks. FIG. 23A is a graph illustrating a temporal change in luminance of the cheek images (that is, the waveform of a pulse wave). Referring to FIG. 23A, p1 denotes each peak in the waveform of the pulse wave, and p2 denotes each inflection point in the waveform of the pulse wave. FIG. 23B is a graph illustrating the first derivatives of the temporal change in luminance of the cheek images.

In such a case, the blood pressure measurement device may calculate pulse wave timings on the basis of, for example, the inflection points p2 illustrated in FIG. 23A. The inflection points in the waveform of the pulse wave correspond to respective local minimum points of the first derivatives of the waveform of the pulse wave as illustrated in FIG. 23B. The use of feature points other than peaks in calculation of pulse wave timings implements more robust pulse-wave-timing calculation.

In addition, peak intervals may be searched for, for example, in a range from 1100 ms to 333 ms on the basis of the general knowledge about pulse waves (e.g., from 60 bpm to 180 bpm). This configuration implements more robust pulse-wave-timing calculation in the usual environment.

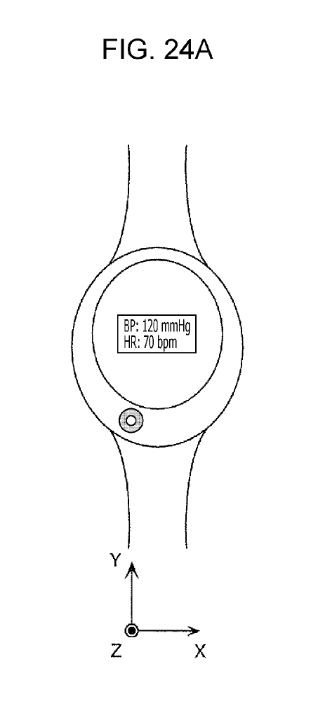

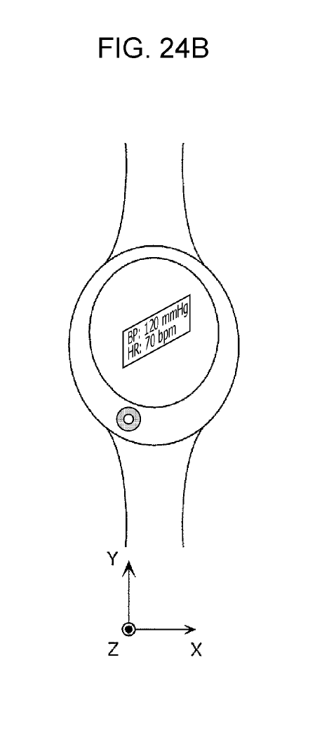

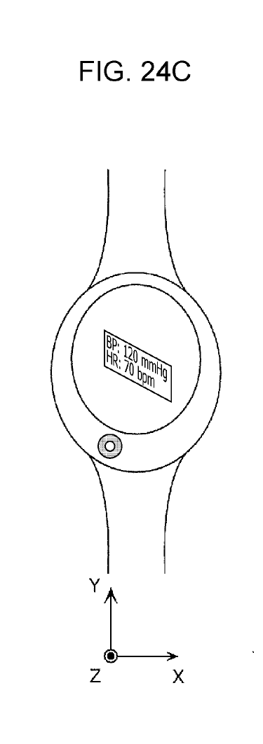

The information displayed on the display 15 in the embodiments described above is merely an example, and how the information is displayed is not limited to this example. Since the blood pressure measurement device 100 is worn on the user's wrist, the size of the display surface of the display 15 is limited and it may be difficult to display lots of information on the display 15. Accordingly, for example, information illustrated in FIGS. 24A to 24C may be displayed instead of the information illustrated in FIG. 17A to 17C. FIGS. 24A to 24C each illustrate a display example of blood pressure and pulse rate.

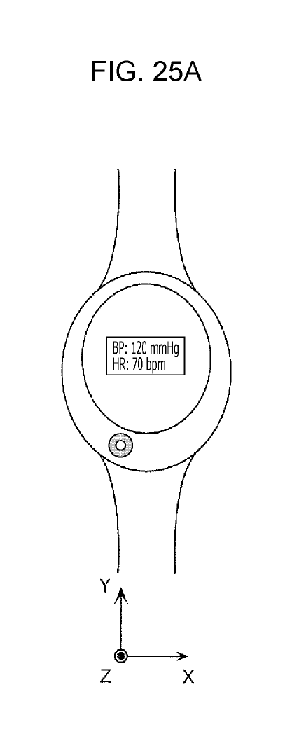

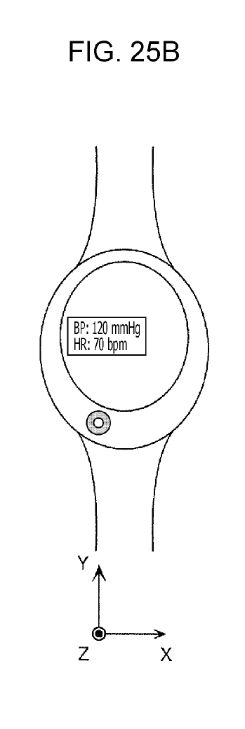

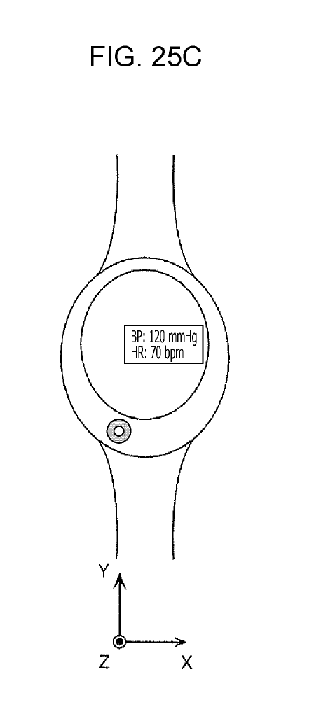

When the image capturing region is shifted to the left or right, the blood pressure measurement device displays information such that the information is tilted leftward or rightward in the embodiments described above as illustrated in FIGS. 17A and 17C; however, how the information is displayed is not limited to this one. For example, the blood pressure measurement device may display the information such that the display position of the information is shifted to the left or right as illustrated in FIGS. 25A to 25C. Even in this case, the user moves their face or wrist to see the information. Thus, the blood pressure measurement device can capture images of the appropriate image capturing region. In addition, if the user's face and the blood pressure measurement device are located far from each other, the font size of the information may be decreased as illustrated in FIGS. 25A to 25C. Since the user consequently brings their face and the blood pressure measurement device closer to each other in order to see the information, more appropriate images can be captured.

The camera 11 is used to determine the first pulse wave timing in the embodiments described above; however, a light meter or the like may be used.

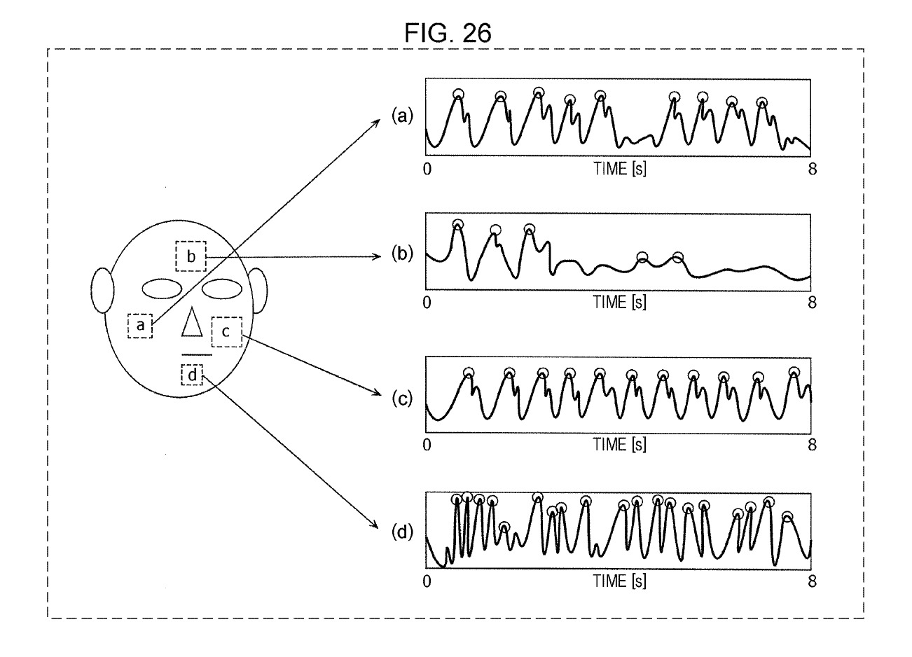

The first pulse wave timing is calculated by using the luminance value in the cheek region of images of the face in the embodiments described above; however, the region is not limited to the cheek region. For example, the luminance value in the forehead region or the chin region may be used to calculate the first pulse wave timing. For example, a given combination of the forehead, cheek, and chin regions may be used to calculate the first pulse wave timing. For example, a region with which a peak is detected most easily in the waveform of the pulse wave among the cheek, forehead, and chin regions may be used to calculate first pulse wave timing. For example, as illustrated in FIG. 26, the blood pressure measurement device may calculate the first pulse wave timing from one of a plurality of regions (of the forehead, cheeks, and chin in this example) of the face. The accuracy of the calculated pulse wave timing changes depending on the skin tone and/or the luminous intensity or the like at the part of the face. Accordingly, by using the most accurate pulse wave timing among pulse wave timings calculated from the plurality of regions to estimate blood pressure, the blood pressure estimation accuracy can be improved. For example, referring to FIGS. 26(a) to 26(d), pulse wave timings having a peak-to-peak time difference of 0.6 seconds or longer and the largest number of peaks, that is, pulse wave timings illustrated in FIG. 26(c), are used in estimation of blood pressure.

The blood pressure estimation method that uses differential pulse transit time is not limited to the estimation method described in the above embodiments. For example, a set of feature points (i.e., first pulse wave timings) may be extracted from each of the plurality of regions, and any set of feature points among the extracted sets of feature points may be used in estimation of the blood pressure. At that time, the blood pressure measurement device may perform calculation of (Equation 2) by using the coefficients α and β corresponding to the region used for estimation. The coefficients α and β may be determined empirically or experimentally in advance for each of the plurality of regions.

In addition, the differential pulse transit time may be corrected in accordance with the region used for estimation. The differential pulse transit time DPTT is denoted by (Equation 3).

DPTT=PTT_{wrist}−PTT_{face} (Equation 3)

That is, a difference between the pulse transit time PTT_{wrist} from the heart to the wrist and the pulse transit time PTT_{face} from the heart to the face corresponds to the differential pulse transit time DPTT.

For example, since a region b is located higher than a region a by approximately 10 cm in FIG. 26, PTT_{face} for the region b is larger than that of the region a and consequently DPTT for the region b is smaller than that of the region a. Accordingly, in the case where blood pressure is estimated by using pulse wave timings calculated from the region b, the blood pressure measurement device may correct the differential pulse transit time by subtracting a predetermined time (e.g., 5 ms) from DPTT. On the other hand, a region d is located lower than the region a by approximately 6 cm. Thus, when the region d is used, the differential pulse transit time may be corrected by adding a predetermined time (e.g., 3 ms) to DPTT.

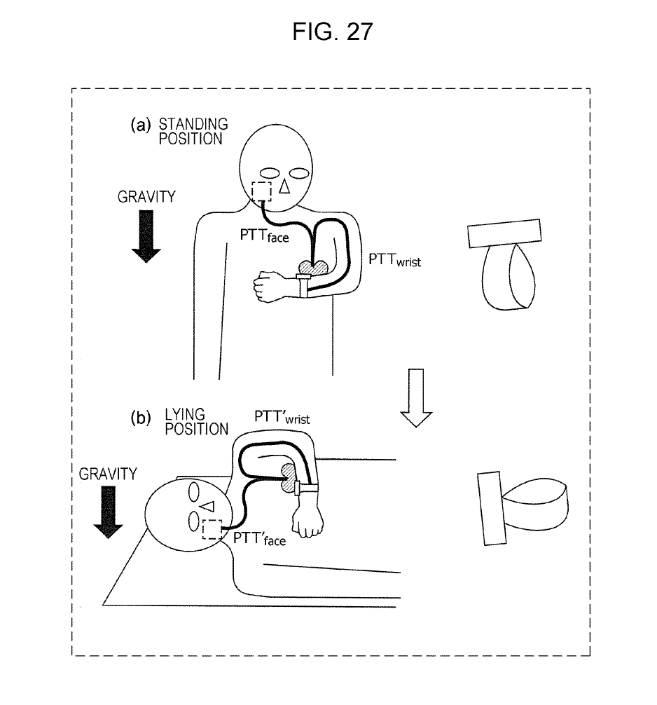

The embodiments described above assume blood pressure measurement in a sitting position and a standing position; however, the body position is not limited to these positions. For example, blood pressure measurement may be performed in a lying position. In such a case, correction may be performed on the differential pulse transmit time DPTT in accordance with the body position. In a sitting position and a standing position, PTT_{face} is affected by the gravity but PTT_{wrist} is hardly affected by the gravity as illustrated in FIG. 27(a).

In a lying position, PTT′_{face} not affected by the gravity as illustrated in is FIG. 27(b). Thus, PTT_{face} is larger than PTT′_{face} (PTT_{face}>PTT′_{face}). On the other hand, PTT′_{wrist} is affected by the gravity because a pulse propagation route from the heart to the arm includes a portion located higher than the heart. Thus, PTT_{wrist} is smaller than PTT′_{wrist} (PTT_{wrist}<PTT′_{wrist}). Accordingly, the differential pulse transit time DPTT increases in a lying position, compared with that in a standing position and a sitting position. For this reason, the blood pressure measurement device corrects the differential pulse transit time DPTT by subtracting a predetermined time (e.g., 20 ms) from DPTT.

For example, a gyro sensor included in the blood pressure measurement device may be used to determine which of a sitting position, a standing position, and a lying position the user is in. The blood pressure measurement device determines that the user is in a sitting position or a standing position when the display surface of the display 15 faces up (90 degrees±10 degrees) and determines that the user is in a lying position when the display surface of the display 15 faces the side (0 degrees+10 degrees) on the basis of the tilt detected by the gyro sensor.

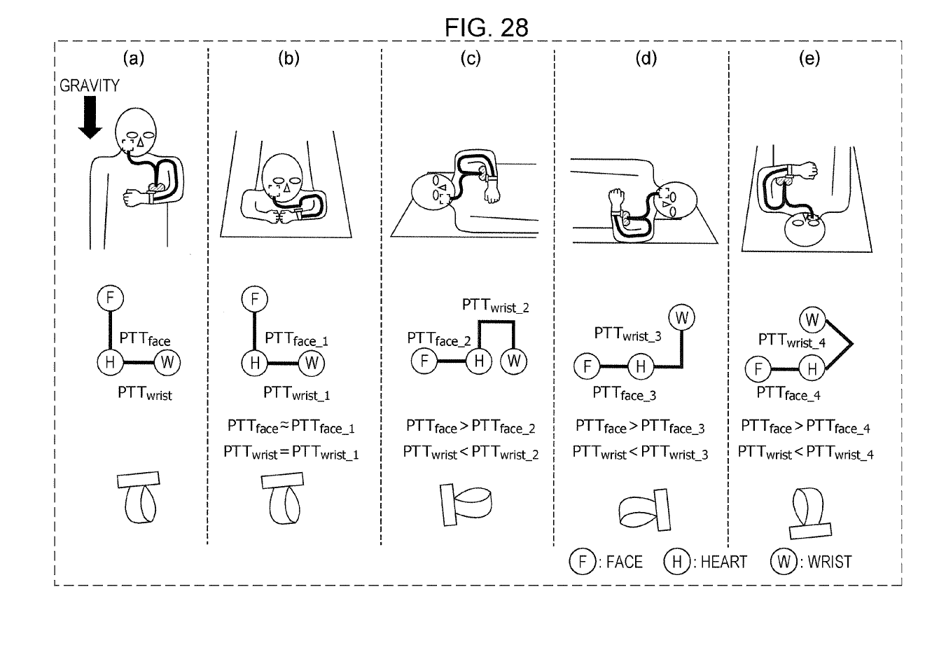

A plurality of lying positions may be determined. FIG. 28 is a diagram for describing correction performed on the differential pulse transit time in accordance with the user's body position. FIG. 28(a) illustrates the case where the user is in a sitting position or a standing position. FIG. 28(a) is used as a reference herein.

FIG. 28(b) illustrates a state where the user is in a prone position. In FIG. 28(b), the user is lying but raises their face when seeing the blood pressure measurement device. Accordingly, the relative position of the face with respect to the heart is slightly lower compared with the case of FIG. 28(a) but the influence of the gravity does not differ much. Thus, differential pulse transit time DPTT_1 in FIG. 28(b) is substantially equal to the differential pulse transit time DPTT in FIG. 28(a).

FIG. 28(c) illustrates the same position as that illustrated in FIG. 27(b). Thus, differential pulse transit time DPTT_2 in FIG. 28(c) is larger than the differential pulse transit time DPTT in FIG. 28(a) as described above. Accordingly, the blood pressure measurement device corrects the differential pulse transit time by subtracting a predetermined time (e.g., 20 ms) from the calculated DPTT_2.

In FIG. 28(d), the left arm is located lower than the heart but the left wrist is located higher than the heart. Differential pulse transit time DPTT_3 in FIG. 28(d) is larger than DPTT in FIG. 28(a). Accordingly, the blood pressure measurement device corrects the differential pulse transit time by subtracting a predetermined time (e.g., 30 ms) from the calculated DPTT_3.

FIG. 28(e) illustrates a state where the user is in a spine position. In FIG. 28(e), the wrist is usually located higher than the heart. Thus, differential pulse transit time DPTT_4 in FIG. 28(e) is larger than the differential pulse transit time DPTT in FIG. 28(a). Accordingly, the blood pressure measurement device corrects the differential pulse transit time by subtracting a predetermined time (e.g., 30 ms) from the calculated DPTT_4, for example. The user's body positions are successfully distinguished from one another on the basis of the tilt of the blood pressure measurement device.

Blood pressure measurement is performed every time the user sees the camera 11 in the embodiments described above; however, the configuration is not limited to this one. For example, the blood pressure measurement device may usually function as a wristwatch and may perform blood pressure measurement upon receipt of a predetermined gesture input (swinging the arm twice, for example).

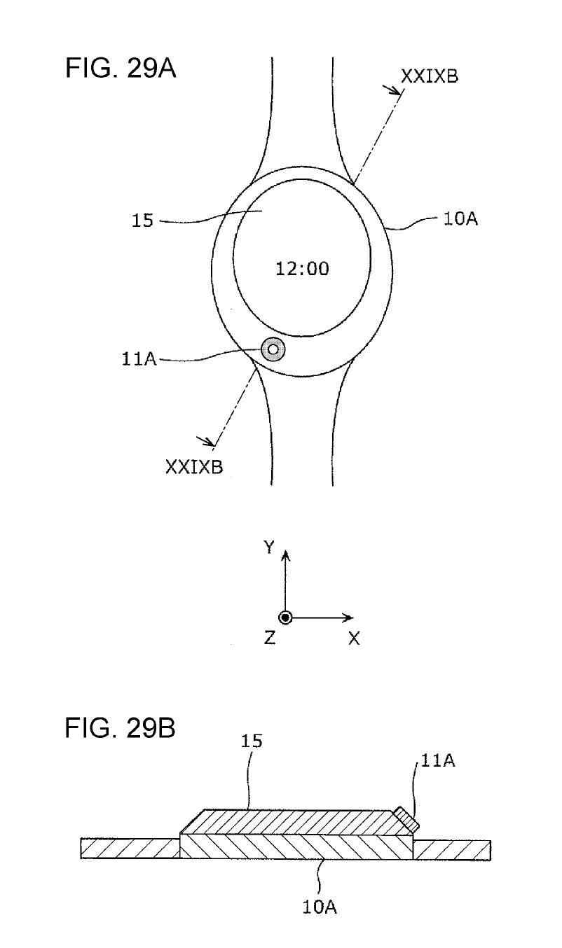

The camera 11 is disposed on a surface that is parallel to the display surface of the display 15 in the embodiments described above; however, the configuration is not limited to this one. For example, a slope portion may be provided around the display 15 on a casing 10A where a camera 11A is disposed as illustrated in FIGS. 29A and 29B. The camera 11A may be disposed on the slope portion at a position that is shifted from the lower side of text displayed on the display 15 by a predetermined angle (from 4 degrees to 23 degrees, for example) clockwise. This configuration makes it easier to attach the camera 11A to the casing 10A and can reduce the assembly work load.

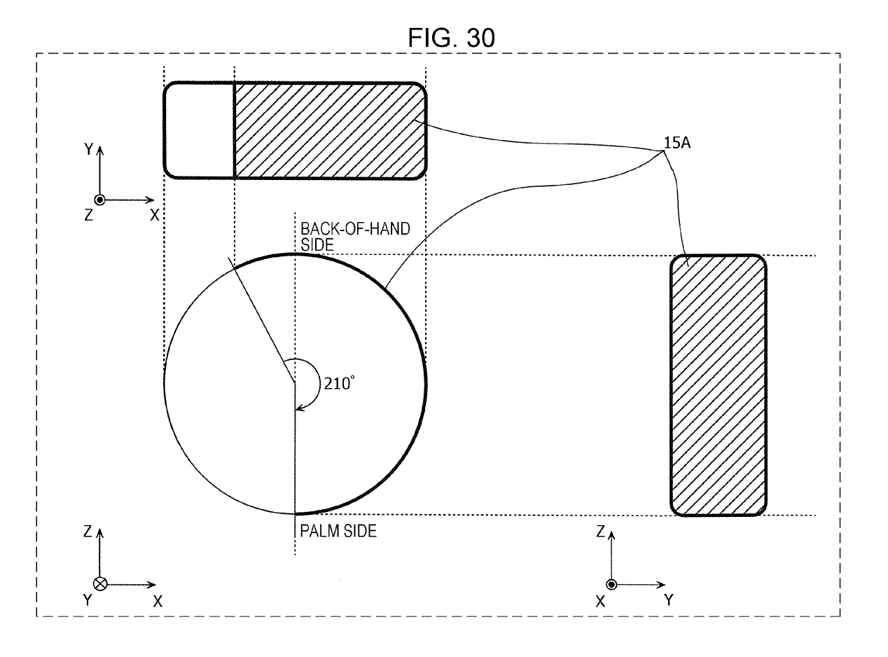

The display 15 is a circular flat-surface display in the embodiments described above; however, the display 15 is not limited to this type. For example, the display 15 may be a quadrature flat-surface display. In addition, the display 15 may be a curved screen display 15A as illustrated in FIG. 30. In this case, the camera 11 is disposed on the display so as to be tilted toward the left side of the displayed text by a predetermined angle (e.g., any angle ranging from 4 degrees to 23 degrees) with respect to the direction of the normal to the display.

Note that a range in which the user can see the display or the camera disposed on the display is limited in this case. Accordingly, the range of the display may be limited. For example, the display 15A or the camera disposed on the display 15A may be disposed in a range from −30 degrees to 180 degrees in the circumferential direction as illustrated in FIG. 30.

The camera 11 is disposed on the lower left side of the display 15 in the embodiments described above; however, the position of the camera 11 is not limited to this position. For example, the camera 11 may be disposed at the positions illustrated in FIGS. 1A and 1B. In this case, images of the user's cheek can be captured as in the embodiments if the optical axis of the camera 11 is directed toward a direction suitable for the disposed position of the camera 11.

The blood pressure measurement device 200 determines whether the user is backlit in the second embodiment; however, the blood pressure measurement device 200 may issue an instruction simply on the basis of the luminance value in the cheek region. For example, when the luminance value in the cheek region is greater than a predetermined upper-limit luminance threshold or is less than a predetermined lower-limit luminance threshold, the blood pressure measurement device 200 may issue an instruction for moving at least one of the user's face and the blood pressure measurement device 200.

The information content displayed on the display 15 is changed in order to change the distance between the user and the blood pressure measurement device in the embodiments described above; however, the configuration is not limited to this one. For example, the blood pressure measurement device may emit a scent to guide the user. If it is desired that the user and the blood pressure measurement device are brought closer to each other, the blood pressure measurement device may extract a scent which the user is fond of in accordance with the distance from the user so as to cause the user to bring the blood pressure measurement device attached their arm closer. On the other hand, if it is desired that the user and the blood pressure measurement device are brought farther from each other, the blood pressure measurement device may extract a scent which the user dislikes.

The image capturing unit is fixed to the blood pressure measurement device so as to be tilted in order to capture images of the user's cheek in the embodiments of the present disclosure; however, the configuration is not limited to this one. A mask may be applied to captured images in order to acquire images of the user's cheek region. For example, feature points such as the eyes, the ears, and the nose may be recognized in images of the face captured by the image capturing unit, and a pulse wave may be extracted on the basis of a change in luminance of data of a skin portion that is left after deletion of data of the feature points. With this configuration, the user's pulse wave can be extracted from captured images of a skin portion even if the entire cheek of the user is not included in the captured images. In addition, the cheek region may be limited by using a physical mask. For example, when the user wears the blood pressure measurement device on their left arm, a mask may be put on the right half of the image capturing unit when viewed from the user. With this configuration, since the amount of information that is not used in captured images decreases, pulse wave components can be acquired more accurately.

All or some of the units or devices, or all or some of the functional blocks of the block diagram illustrated in FIG. 6 may be implemented by one or one or more electronic circuits including a semiconductor device, a semiconductor integrated circuit (IC), or a large scale integration (LSI) in embodiments of the present disclosure. The LSI or IC may be implemented by one chip or may be implemented by a combination of a plurality of chips. For example, functional blocks other than the storage element may be integrated on one chip. Although the term “LSI” or “IC” is used herein, the name changes depending on the degree of integration and the term “system LSI”, “very large scale integration (VLSI)”, or “ultra large scale integration (ULSI)” may be used. A field programmable gate array (FPGA) that is programmable after production of the LSI or a reconfigurable logic device in which connections of elements in the LSI are reconfigurable or setup of circuit cells in the LSI are possible may be used for the same purpose.

Further, all or some of functions or operations of the units, the apparatuses, and part of the apparatuses can be implemented by software-based processing. In this case, the software is stored on one or one or more non-transitory recoding media, such as a ROM, an optical disc, or a hard disk drive. When the software is executed by a processing device (processor), the software causes the processing device (processor) and its peripheral devices to carry out a specific function included in the software. A system or apparatus may include one or one or more non-transitory recording media storing the software, the processing device (processor), and necessary hardware devices, for example, an interface.

In addition, an embodiment of the present disclosure may be a computer system including a microprocessor and a memory. The memory may store the computer program, and the microprocessor may execute the computer program.

In addition, the program or digital signals may be transferred to another independent computer system after being recorded on a recording medium or via a network and may be executed by the independent computer system.

In addition, each of the components of the embodiments may be implemented by dedicated hardware or by executing a software program suitable for the component. Each of the components may be implemented as a result of a program executor, such as a CPU or a processor, reading and executing a software program stored on a recording medium, such as a hard disk or a semiconductor memory.

The embodiments of the present disclosure can be used as wristwatch-type blood pressure measurement devices.

What is claimed is:

1. A blood pressure measurement device comprising:
a bracelet that comes into contact with a wrist of a user, the bracelet having an annular shape and having an outer surface and an inner surface;
a display that is disposed on the outer surface of the bracelet and that includes a display surface;
an image capturing device that is disposed on the outer surface of the bracelet and that captures a plurality of images of the user, the image capturing device having an optical axis that is tilted with respect to a direction of a normal to the display surface of the display;
a pulse wave detector that is disposed on the inner surface of the bracelet and that detects a pulse wave at the wrist of the user; and
a processing circuit that estimates a blood pressure of the user,
wherein the processing circuit
calculates a first pulse wave timing from a temporal change in luminance value in a cheek region of the user in the plurality of images,
determines a second pulse wave timing from the pulse wave detected by the pulse wave detector, and
estimates a blood pressure of the user from a time difference between the first pulse wave timing and the second pulse wave timing.

2. The blood pressure measurement device according to claim 1,
wherein the display surface of the display has a top-bottom direction and a left-right direction, and
wherein when viewed from the top-bottom direction of the display surface of the display, the optical axis of the image capturing device is tilted in the left-right direction of the display surface of the display with respect to the direction of the normal.

3. The blood pressure measurement device according to claim 1, wherein the processing circuit further
determines the cheek region of the user in the plurality of images of the user, and
calculates the first pulse wave timing in accordance with a temporal change in luminance value in the determined cheek region.

4. The blood pressure measurement device according to claim 3, wherein the processing circuit further
determines whether the cheek region of the user is successfully determined in the plurality of images of the user,
calculates a relative position of a face of the user with respect to the image capturing device by using the plurality of images of the user upon failing to determine the cheek region of the user, and
displays on the display an instruction for changing a positional relationship between the face of the user and the image capturing device in accordance with the relative position of the face of the user.

5. The blood pressure measurement device according to claim 4, wherein the processing circuit calculates the relative position of the face of the user on the basis of a size and a position of at least one of an eye, an ear, and a nose of the user in the plurality of images of the user.

6. The blood pressure measurement device according to claim 5, wherein the processing circuit further
selects a recognition model, from among a plurality of recognition models used to recognize at least one of the eye, the ear, and the nose of the user in images, on the basis of which of a right wrist and a left wrist of the user the blood pressure measurement device is worn on and which of a palm side and a back-of-hand side of the wrist the blood pressure measurement device is worn on, and
recognizes at least one of the eye, the ear, and the nose of the user in the plurality of images of the user by using the selected recognition model.

7. The blood pressure measurement device according to claim 6, wherein the processing circuit determines which of the palm side and the back-of-hand side the blood pressure measurement device is worn on, on the basis of a temporal change in position of at least one of the eye, the ear, and the nose of the user in the plurality of images of the user.

8. The blood pressure measurement device according to claim 7, wherein the processing circuit
calculates a distance and an orientation of the face of the user with respect to the image capturing device on the basis of the sizes and positions of the eye, the ear, and the nose of the user in the plurality of images of the user, and
displays on the display at least one of an instruction for twisting the wrist and an instruction for bending or stretching an elbow in accordance with the calculated distance and orientation.

9. The blood pressure measurement device according to claim 1, wherein the processing circuit controls, in accordance with a relative position of the face of the user, at least one of a display angle, a display position, and a display size of information displayed on the display.

10. The blood pressure measurement device according to claim 8, wherein the processing circuit reduces the display size of the information displayed on the display when the distance of the face of the user with respect to the image capturing device is greater than a threshold distance.

11. The blood pressure measurement device according to claim 1, wherein the processing circuit further
determines whether the user is backlit on the basis of luminance values of the plurality of images of the user, and
displays on the display an instruction for moving at least one of the image capturing device and the face of the user upon determining that the user is backlit.

12. The blood pressure measurement device according to claim 11, wherein the processing circuit displays on the display an instruction for moving the blood pressure measurement device to an upper position upon determining that the user is backlit.

13. The blood pressure measurement device according to claim 11, wherein the processing circuit displays on the display an instruction for twisting a body of the user upon determining that the user is backlit.

14. A blood pressure estimation device comprising:
a bracelet that comes into contact with a wrist of a user, the bracelet having an annular shape and having an outer surface and an inner surface;
a display that is disposed on the outer surface and that includes a display surface;
an image capturing device that is disposed on the outer surface and that captures images of the user at different times, the image capturing device having an optical axis that is tilted with respect to a direction of a normal to the display surface, the image capturing device including a first pixel outputting a first light intensity value upon receiving first light parallel to the optical axis, the first pixel outputting a second light intensity value upon receiving second light not parallel to the optical, the first value being bigger that the second value;
a pulse wave detector that is disposed on the inner surface and that detects a pulse wave at the wrist of the user; and
a processing circuit that estimates a blood pressure of the user based on a time difference between a first pulse wave timing and a second pulse wave timing,
wherein the processing circuit determines the first pulse wave timing being a first time point at which a waveform indicates a first local maximum,
wherein the processing circuit determines the waveform indicating luminance values at a cheek of the user in the images at the times, and
wherein the processing circuit determines the second pulse wave timing being a second time point at which the pulse wave indicates a second local maximum.
